SEQUENCE LISTING (13 sequences)

SEQ ID NO: 1 - DNA, 26 bp, Homo sapiens
ggaagcagaccacgtggtctgcttcc

SEQ ID NO: 2 - DNA, 68 bp, Homo sapiens
agcaggtgttgggaggcagcaggtgttgggaggcagcaggtgttgggaggcagcaggtgttgggaggc

SEQ ID NO: 3 - DNA, 41 bp, Homo sapiens
ggccgatgggcagatagagggggccgatgggcagatagagg

SEQ ID NO: 4 - DNA, 17 bp, Homo sapiens
cggacaactgttgaccg

SEQ ID NO: 5 - DNA, 17 bp, Homo sapiens
cggaggactgtcctccg

SEQ ID NO: 6 - DNA, 17 bp, Homo sapiens
cggagtactgtcctccg

SEQ ID NO: 7 - DNA, 20 bp, Homo sapiens
tactgtatgtacatacagta

SEQ ID NO: 8 - DNA, 21 bp, Homo sapiens
gaattgtgaggctcacaattc

SEQ ID NO: 9 - DNA, 42 bp, Homo sapiens
tcgagtttaccactccctatcagtgatagagaaaagtgaaag

SEQ ID NO: 10 - DNA, 12 bp, Homo sapiens
taatgatgggcg

SEQ ID NO: 11 - DNA, 12 bp, Homo sapiens
ataggccgggcc

SEQ ID NO: 12 - DNA, 85 bp, Homo sapiens
cggagtactgtcctccgcggagtactgtcctccgcggagtactgtcctccgcggagtactgtcctccgcggagtactgtcctccg

SEQ ID NO: 13 - DNA, 51 bp, Homo sapiens
cggagtactgtcctccgcggagtactgtcctccgcggagtactgtcctccg

(19) United States
(12) Patent Application Publication
(10) Pub. No.: US 2002/0137699 A1
(43) Pub. Date: Sep. 26, 2002

(54) EXPRESSION SYSTEMS COMPRISING CHIMERIC PROMOTERS WITH BINDING SITES FOR RECOMBINANT TRANSCRIPTION FACTORS

(76) Inventors: ROLF MUELLER, MARBURG (DE); DIRK NETTELBECK, MARBURG (DE); HANS-HARALD SEDLACEK, MARBURG (DE)

Correspondence Address: HELLER, EHRMAN, WHITE & McAULIFFE, 815 Connecticut Avenue, Suite 200, Washington, DC, 20006, US

(21) Appl. No.: 09/352,767

(22) Filed: Jul. 14, 1999

(30) Foreign Application Priority Data
Jul. 14, 1998 (DE) 198 31 420.5

(51) Int. Cl. A61K 48/00; C12N 15/87; C07H 21/04

(52) U.S. Cl. 514/44; 536/23.2; 536/23.1; 435/320.1; 800/21; 435/455

(57) ABSTRACT

Expression systems comprising chimeric promoters with binding sites for recombinant transcription factors The present invention relates to a nucleic acid construct which comprises the following components:

Component a): at least one promoter

Component b): a nucleic acid sequence encoding at least one recombinant transactivator whose transcription is activated by component a) and which comprises:

component b1): a nucleic acid sequence encoding a DNA-binding domain

component b2): a nucleic acid sequence encoding a transactivation domain comprising glutamine, serine and threonine

Component c): at least one a nucleic acid sequence sequence for binding the expression product of component b)

Component d): at least one promoter which comprises the CDE-CHR element or the E2FBS-CHR element and whose 5′ end is bound, i.e., linked, to the 3′ end of component c)

Component e): at least one effector gene whose transcription is activated by the expression product of component b) binding to component c);

to its preparation and its use; to vectors comprising the nucleic acid construct, cells comprising these vectors, and to the use of the nucleic acid construct for the preparation of a medicament.

### BACKGROUND OF THE INVENTION

Gene transcription is governed by activation sequences (promoters and enhancers). Such activation sequences represent nucleotide sequences to which transcription factors bind, thus arranging transcription of the corresponding gene.

There are now known a large number of activation sequences as promoters or enhancer sequences.

Depending on their function or origin, they are divided into universal activation sequences, i.e. activation sequences which are effective in any cell, into activation sequences of viral origin, and in activation sequences with a limited action.

The limitation can be, for example, cell-specific, metabolic (for example under hypoxic conditions) or cell-cycle-specific.

These limitations regarding the function of the activation sequences are exploited in the directed expression of a structural gene, for example for the purposes of gene therapy. Thus, it is a current technology to put the expression of a structural gene under the control of a cell-specific promoter (Sikora, Trends Biotech. 11, 197 (1993)).

However, in many cases it does not suffice to limit the transcription of an effector gene by a cell-specific promoter, partly because activation of the cell-specific promoter is not cell-specific enough, partly because expression of the effector gene is only desired in those cells of the selected cell type which are in a particular functional state. Such a functional state may be, for example, the cell-cycle phase of the cell.

To regulate the expression of an effector gene more extensively, it is therefore desirable to employ, for controlling expression of a structural gene, a plurality of promoters with different specificities.

The chimeric promoter technology (Patent Applications, for example, PCT/GB95/02000, EP-A 0 790 313) was developed for combining a promoter of any specificity with a cell-cycle-specific promoter. This technology consists in linking an upstream promoter of any specificity with the downstream CDE-CHR element or the E2FBS-CHR element.

Cell division is divided into the consecutive phases G0 or G1, S, G2 and M. The S phase is the phase of DNA synthesis, it is followed by the transition phase G2 (G2 phase), followed by the mitotic phase (M phase), in which a mother cell divides into two daughter cells. Between the M phase and the S phase there is the resting phase GO (GO phase) or the transition phase G1 (G1 phase).

Cell division is driven by a group of protein kinases, the cyclin/cdk complexes. These are composed of a catalytic subunit [cyclin dependent kinase (cdk, for example cdk-1,-2,-3,-4,-5,-6,-7 or -8) and a regulatory subunit, cyclin (for example cyclin A, -B1-B3,-D1-D3,-E, -H or -C].

Different cdk complexes are particularly active in each cell cycle phase, thus,

| | |
|---|---|
| in the middle G1 phase | cdk4/cyclin D1-3 and |
| | cdk6/cyclin D1-3 |
| in the late G1 phase | cdk2/cyclin E |
| in the S phase | cdk2/cyclin A |
| in the G2/M transition phase | cdk1/cyclin B1-3 and |
| | cdk1/cyclin A |

The activity of the cyclin/cdk complexes consists in the phosphorylation and thus activation or inactivation of proteins which play a direct or indirect role in the control of DNA synthesis and mitosis.

Corresponding to their function in the cell cycle, the genes for some cyclins and cdks are transcribed periodically and/or activated or inhibited periodically, for example by the regulated degradation of cyclins, by the cell-cycle-phase-specific binding of inhibitors (for example p16INK4A, p15INK4B, p21Cip1, p27Kip1, p18INK4C, p19INK4D, p57) or by modification by activating (for example by the cdc25 phosphatases, such as cdc25A, cdc25B and cdc25C or cdk7/cyclin H) or inhibiting (for example weel kinase) enzymes (see overview by Zwicker and Müller, Progr. Cell Cycle Res. 91 (1995); La Thangue, Curr Opin. Cell Biol. 443 (1994); MacLachlan et al., Crit. Rev. Eukaryotic Gene Expr. 127 (1995)).

The periodic expression of cdc25C in the G2 phase of the cell cycle is essentially governed by an element (CDE-CHR) in the promoter region of the gene for cdc25C. This CDE-CHR element is occupied by a repressing protein in the G0/G1 phase and free in the G2 phrase. The nucleotide sequence of this promoter element was identified and, equally, also found in the promoters of the genes for cyclin A and cdk-1, while a nucleotide sequence which was found in the promoter for B-myb was different in parts (E2FBS-CHR). A study of the cell-cycle-dependent function of these promoter elements demonstrated that their blockage in the G0/G1 phase is followed by an upregulation of the transcription of the gene in question, which takes place particularly early (in the middle G1 phase) in the case of the B-myb gene, in the G1/S transition phase in the case of cyclin A, in the S phase in the case of cdk-1 gene and only in the late S phase in the case of the cdc25C gene (Zwicker and Müller, Progr. Cell Cycle Res. 91 (1995); Lucibello et al., EMBO J. 132 (1995); Liu et al., Nucl. Acids Res. 2905 (1995); Zwicker et al., Nucl. Acids Res. 3822 (1995); EMBO J. 4514 (1995)).

Surprisingly, it has also been found that the element CDE-CHR (of the promoter for the cyclin 25C, cyclin A and cdk-1 gene) and the element E2FBS-CHR (of the promoter for the B-myb gene) is capable of inhibiting not only activation and transcription of the homologous genes in the G0/G1 phase, but also the activation and transcription of other genes. This invention led to Patent Applications PCT/GB95/02000, EP-A 0 777 739, EP-A 0 777 740, EP-A 0 804 601, EP-A 0 807 183, EP-A 0 790 313 and EP-A 0 860 445.

These patent applications disclose the combination of a cell-cycle-dependent promoter with an unspecific, cell-specific, virus-specific or metabolically activatable promoter for the regulated activation of the transcription of an effector gene which encodes a protein for the prophylaxis and/or therapy of a disease. Examples of such diseases may be tumor diseases, leukemias, autoimmune diseases, various types of arthritis, allergies, inflammations, rejections of transplanted organs, diseases of the blood circulation system, of the blood clotting system, infections or damages to the central nervous system.

The so-called chimeric promoter was developed for combining various promoters with a cell-cycle-specific element. In this chimeric promoter, the activity of an unspecific, cell-specific, virus-specific or metabolically activatable activation sequence (or promoter sequence) by the promoter element CDE-CHR or E2FBS-CHR, which is located immediately downstream, is restricted largely to the cell cycle phases S and G2.

More extensive studies into the function of, in particular, the promoter element CDE-CHR, revealed that regulation, of an upstream activator sequence, which is cell-cycle-dependent due to the CDE-CHR element, depends largely on activation of the activation sequence of transcription factors having high-glutamine activation domains (Zwicker et al., Nucl. Acids. Res. 3822 (1995)).

Such transcription factors include, for example, Oct-2, Sp1 and NF-Y.

As a consequence, it is desirable to combine such transcription factors with the promoter element CDE-CHR for chimeric promoters or the promoter element E2F-BS-CHB of the B-myb gene (Zwicker et al., Nucl. Acids Res. 23, 3822 (1995)).

### SUMMARY OF THE INVENTION

The present invention relates to a nucleic acid construct in which any promoter can be linked with the CDE-CHR element or the E2FBS-CHR element to give a functional chimeric promoter and comprises the following components:

```
Component a)
at least one promoter
Component b)
a nucleic acid sequence, prefereably a DNA sequence, encoding at least one
recombinant transactivator whose expression is activated by component a) and
which comprises
    b1) a nucleic acid sequence, prefereably a DNA sequence encoding a DNA-
    binding domain
    b2) a nucleic acid sequence, prefereably a DNA sequence encoding a
    transactivation domain which comprises and preferably is high in glutamine,
    serine and threonine.
Component c)
at least one a nucleic acid sequence, prefereably a DNA sequence for binding the
expression product of component b)
Component d)
at least one promoter and preferably a minimal (a minimal promoter is a promoter
having at least one transcription activating element) which comprises the CDE—CHR
element or the E2FBS—CHR element and whose 5′ end is bound, i.e., linked, to the
3′ end of component c)
Component e)
at least one effector gene whose transcription is activated by the expression product
of component b) binding to component c).
```

An example of the arrangement of the individual components is shown by way of example in FIG. 1.

The function of the nucleic acid construct according to the invention is such that activation of the cell-specific, metabolically activatable, virus-specific, cell-cycle-specific or universally activatable promoter [component a)] leads to transcription of the gene [component b)] for the recombinant transcription activator which, in turn, binds to its DNA-binding sequence [component c)], and thereby activates the minimal CDE-CHR comprising promoter [component d)], whereby transcription of the effector gene [component e)] is arranged.

In the G0/G1 phase of the cell cycle, the CDE-CHR element of component d) is blocked by binding the so-called CDF protein, whereby activation of the transcription of component e) is inhibited.

The nucleic acid construct according to the invention can be extended in various ways:

Several identical or different effector genes [components e, e′, e″] can be introduced into the nucleic acid construct, these effector genes either being linked to each other via an IRES sequence, or with components c) and d) being added upstream of each effector gene.

Component c) may be added upstream of component a) in such a way that the recombinant transactivator expressed by component b), also causes an enhanced activation of component a) in the sense of a self-enhancing promoter.

Such expression systems are shown, for example, in FIG. 2(A).

Such self-enhancing promoters have already been described in detail in Patent Application EP-A 0 848 061.

In a particular embodiment of the present invention, the self-enhancing promoter system may also be added to the expression system according to the invention, such as shown, for example, in FIG. 2(B).

Component b) can be extended by introducing a component b3) which expresses a protein A which binds to a coupling substance [component f)] and by introducing a component b4) which expresses a protein B which also binds to the coupling substance f), to give a recombinant transactivator [component b′)] which can be controlled by the coupling substance f), i.e. pharmacologically.

Such an extension is shown, for example, in FIG. 3.

The introduction of such a transactivator makes the expression system according to the invention pharmacologically controllable. Such expression systems have already been described in detail in Patent Application EP-A 0 848 061.

A further, progesterone-inducible expression system originates by the combination of component b) and/or component f) with the progesterone receptor ligand binding domain (Wang et al., Gene Therapy 4, 432 (1997)).

The expression system can be governed additionally by introducing the nucleic acid sequence for a binding protein [component b5)] for a cellular regulatory protein between or to components bl) and b2) [component b″ being composed of component b1), b2) and b5)]. This additional governing is caused by the regulatory protein adhering to the binding protein in the normal cell and thus blocking the function of the recombinant transactivator which is expressed by component b″), that is to say the binding of this transactivator to component c). In cells in which the cellular regulatory protein is mutated and can therefore not adhere to the binding protein, or in which the regulatory protein is reduced, is lacking or is bound to cellular, viral, bacterial or parasitic binding proteins which compete with component b5), the recombinant transactivator [component b″)] is functional and capable of binding to component c).

Component b″) is shown in FIG. 4 by way of example.

The introduction of a nuclear localization signal into component b), b′) or b″) allows the operativeness of the expression system according to the invention to be increased.

By combining two or more of the abovementioned extensions.

The effector gene [component e)] encodes a pharmacologically active ingredient selected from the group consisting of cytokines, growth factors, antibodies or antibody fragments, receptors for cytokines or growth factors, proteins with an antiproliferative, apoptotic or cytostatic action, angiogenesis inhibitors, coagulation inhibitors, thrombosis-induced substances and coagulation inhibitors, fibrinolytically active substances, plasma proteins, complement-activating proteins, peptide hormones, virus coat proteins, bacterial antigens and parasitic antigens, and proteins and ribozymes which affect blood circulation. Preferably, the effector gene encodes a ribozyme which inactivates the mRNA which encodes a protein selected from the group consisting of cell cycle control proteins, in particular cyclin A, cyclin B, cyclin D1, cyclin E, E2F1-5, cdc2, cdc25C or DP1 or viral proteins or cytokines or growth factors or receptors of these.

In another embodiment, the effector gene encodes an enzyme which cleaves a prodrug into a pharmacon.

In a further embodiment, the effector gene may encode a ligand effector fusion protein, it being possible for the ligand to be an antibody, an antibody fragment, a cytokine, a growth factor, an adhesion molecule or a peptide hormone, and for the effector to be a pharmacologically active ingredient as described above or an enzyme. For example, the structural gene may encode a ligand enzyme fusion protein where the enzyme cleaves a prodrug into a drug and the ligand binds to a cell surface, preferably to endothelial cells or tumor cells.

The nucleic acid constructs are preferably composed of DNA. The term nucleic acid constructs is to be understood as meaning artificial nucleic acid structures which can be transcribed in the target cells. They are preferably inserted into a vector, plasmid vectors or viral vectors being especially preferred. In a preferred embodiment, these vectors are administered to patients externally or internally, locally, into a body cavity, into an organ, into the blood circulation, into the respiratory tract, into the gastrointestinal tract, into the urogenital tract or intramuscularly or subcutaneously.

The nucleic acid constructs according to the invention allow an effector gene [component e)] to be expressed either cell-specifically, virus-specifically, under certain metabolic conditions and/or cell-cycle-specifically, the effector gene preferably being a gene which encodes a pharmacologically active ingredient or else an enzyme which cleaves an inactive prodrug into an active drug. The effector gene may be chosen in such a way that the pharmacologically active ingredient or the enzyme is expressed as a fusion protein together with a ligand, and this ligand binds to the surface of cells, for example proliferating endothelial or tumor cells.

Another subject of the present invention is cells of yeasts or mammals which comprise a nucleic acid construct according to the invention. In a particularly preferred embodiment, the nucleic acid constructs are introduced into cell lines which can then be used, after transfection, for expressing the transgene. These cells can therefore be used for providing a medicine for patients. A preferred use of the nucleic acid construction according to the invention consists in the treatment of a disease, where providing the medicine encompasses introducing a nucleic acid construct into a target cell and its virus- or target-cell-specific or metabolically specific or unspecific and cell-cycle-specific expression.

The invention furthermore relates to the administration of mammalian cells which comprise a nucleic acid construct according to the invention for the preparation of a medicine for treating a disease. For example, endothelial cells, obtained from blood, may be transfected in vitro with the nucleic acid construct according to the invention and injected to the patient, for example intravenously.

Such in-vitro-transfected cells may also be administered to patients in combination with a vector according to the invention. This combination consists in cells and vectors being administered or injected, in each case simultaneously or at different points in time, to identical or different sites.

The nucleic acid constructs according to the invention do not occur naturally in this form, i.e. the effector gene for the active ingredient or for an enzyme or for a ligand effector fusion protein is not combined in nature with the minimal promoter according to the invention comprising a CDE-CHR element or an E2FBS-CHR element and with a DNA-binding sequence for a recombinant transactivator.

The promoters and the effector gene for the active ingredient (or for the enzyme) of the nucleic acid constructs according to the invention are chosen to suit the intended purpose.

### BRIEF DESCRIPTION OF THE DRAWINGS

FIG. 1 is a shematic representation of examples of nucleic acid constructs.

FIGS. 2A-2B are shematic representations of examples of nucleic acid constructs.

FIG. 3 is a shematic representation of examples of nucleic acid constructs.

FIG. 4 is a shematic representation of examples of nucleic acid constructs.

FIGS. 5(A)-5(D) are schematic representations of examples of RTA constructs and of reporter constructs.

FIGS. 6(A)-6(C) are schematic representations of examples of control constructs as described in section I. Thin lines: pGL3 vector,Promega; bold lines: promoters in the MCS of pGL3.

FIGS. 7(A)-7(C) are schematic representations of examples of RTA constructs as described in Section II, the vector skeleton is derived from pGL3 (Promega).

FIGS. 8(A)-8(B) are schematic representations of examples of reporter constructs as described in Section II

FIGS. 9(A)-9(B) are schematic representations of examples of control constructs as described in Section II. Thin lines: pGL3 vector, Promega; bold lines: promoters in the MCS of pGL3

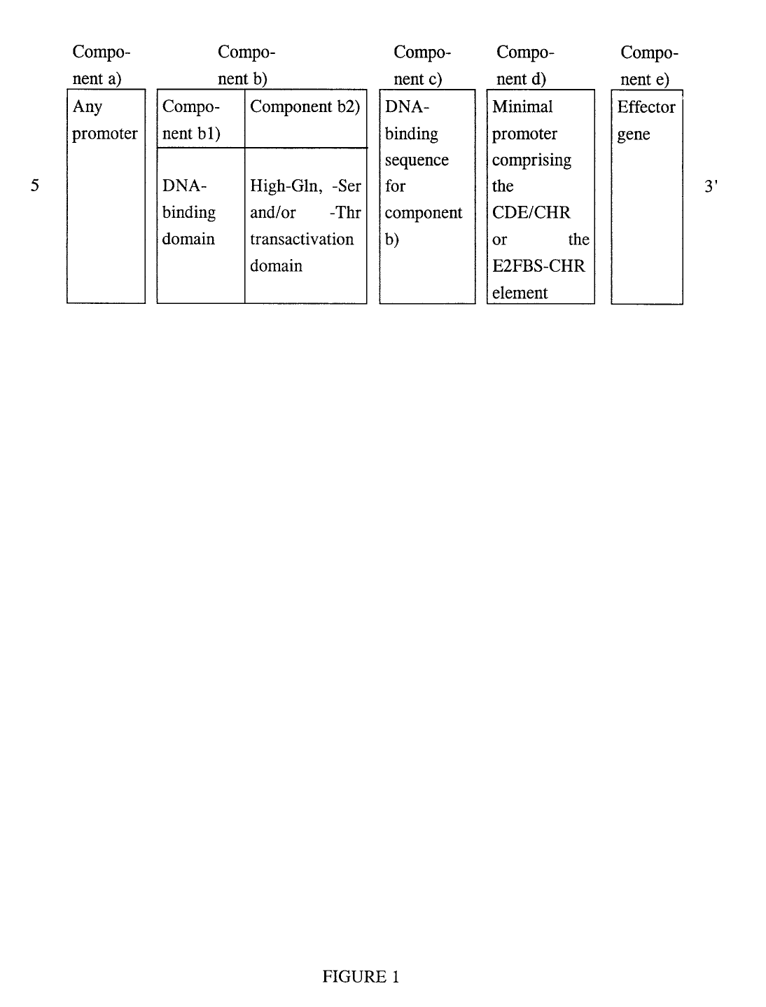

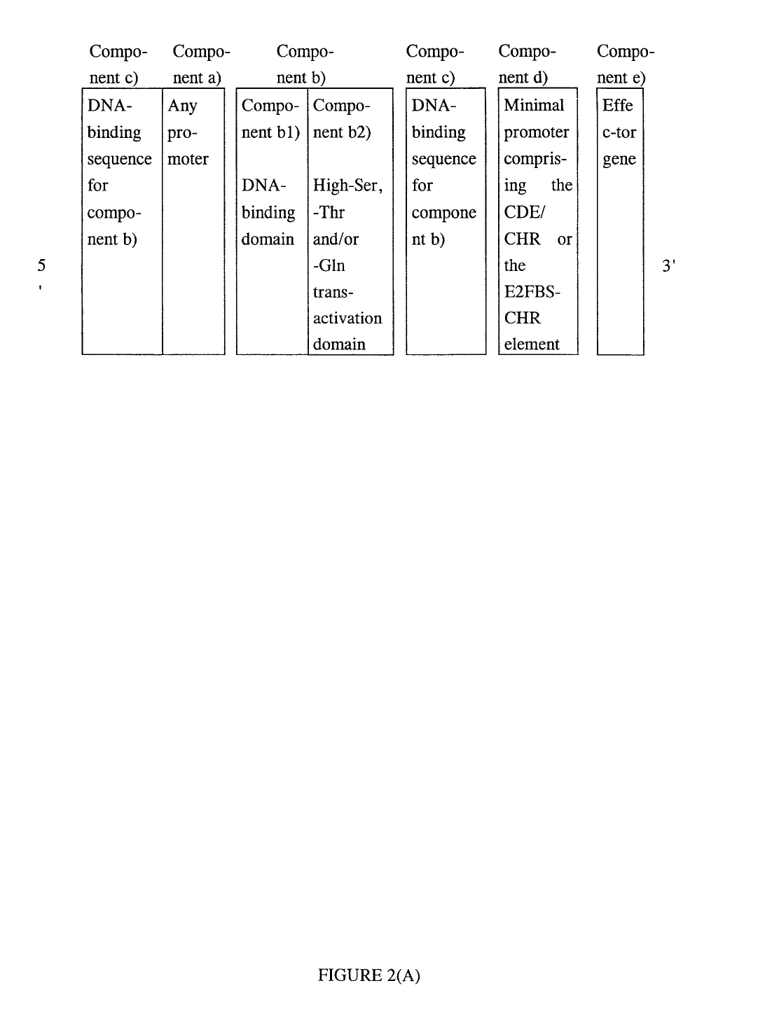

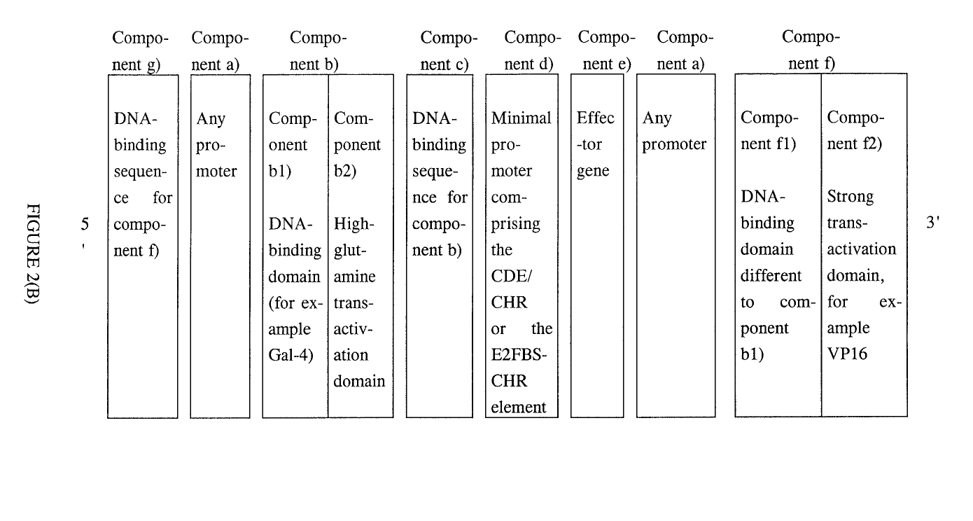

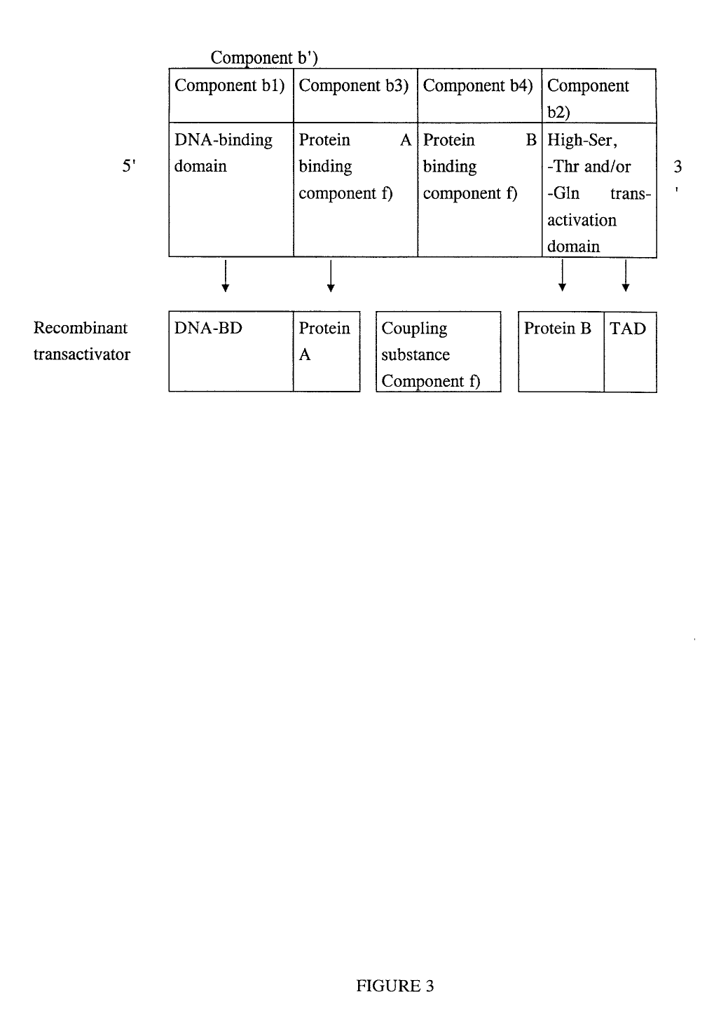

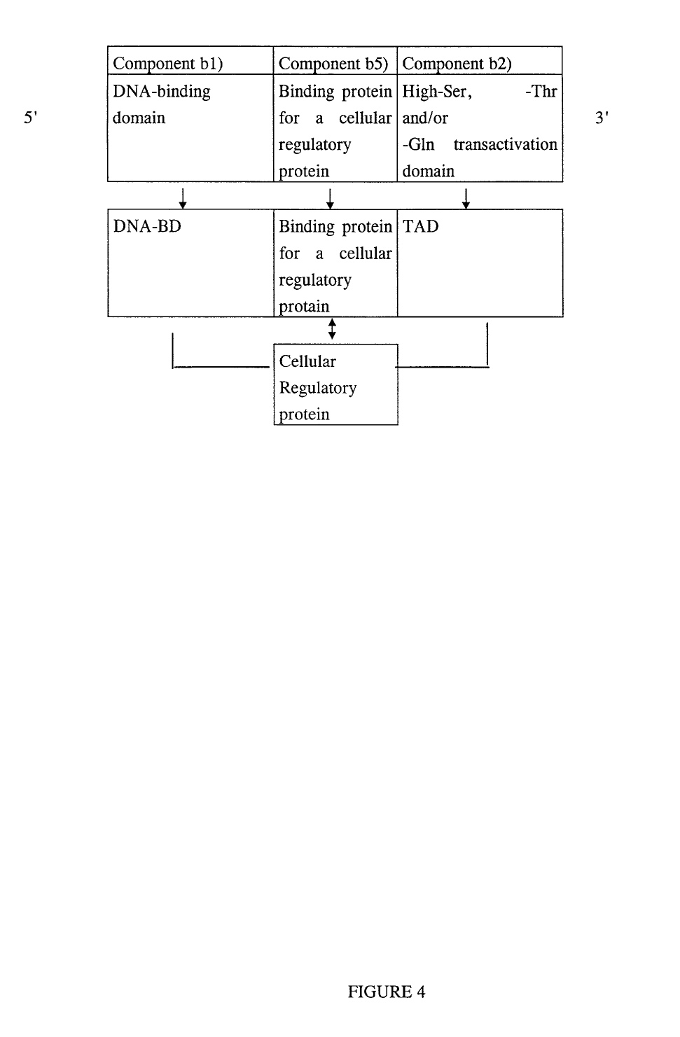

### DETAILED DESCRIPTION OF PREFERRED EMBODIMENTS

I) Promoters [Component a)]

Promoter sequences to be used for the purposes of the invention are nucleotide sequences which, after binding transcription factors, activate the transcription of a structural gene which is adjacent on the 3′-end. The choice of the promoter sequence to be combined with the CDE-CHR or E2FBS-CHR-comprising promoter sequence [component d)] depends on the disease to be treated and the target cell to be transduced. Thus, the additional promoter sequence may be activatable universally, target-cell specifically, under particular metabolic conditions, cell-cycle-specifically or virus-specifically. The promoter sequences to be selected include, for example:

a) Universally activatable promoters and activator sequences such as, for example,

the RNA polymerase III promoter

the RNA polymerase II promoter

the CMV promoter and enhancer

the SV40 promoter and enhancer

b) Viral promoter and activator sequences, such as, for example,

HBV

HCV

HSV

HPV

EBV

HTLV

HIV

When using the HIV promoter, all of the LTR sequence including the TAR sequence [position <−453 to >−80, Rosen et al., Cell 41, 813 (1985)] is to be used as virus-specific promoter.

c) Metabolically activatable promoter and enhancer sequences such as, for example, the hypoxia-inducible enhancer (Semenza et al., PNAS 88, 5680 (1991); McBurney et al., Nucl. Acids Res. 19, 5755 (1991)) or radiation-inducible promoters such as, for example, the egr-1 promoter ionizing-radiation-inducible element (Hallahan et al., Nature Med. 1, 786, 1995)).

d) Cell-cycle-specifically activatable promoters. These are, for example, the promoter of the cdc25C gene, the cyclin A gene, the cdc2 (cdk-1) gene, the Bmyb gene, the DHFR gene or the E2F-1 gene, or else binding sequences for transcription factors which occur, or are activated, during cell proliferation. These binding sequences include monomers or multimers of the nucleotide sequence [5′-GGAAGCAGACCACGTGGTCTGCTTCC-3′; SEQ ID NO: 1]; Blackwood und Eisenmann, Science 251, 1211 (1991)] which is also termed Myc E box.

e) Tetracycline-activatable promoters such as, for example, the tetracycline operator in combination with a suitable repressor.

f) Cell-specifically activatable promoters

These preferably include promoters or activator sequences of promoters or enhancers from those genes which encode proteins preferentially formed in selected cells.

For example, it is preferred, for the purposes of the invention, to use promoters for the following proteins in the following cells:

f1) Promoter and activator sequences activated in endothelial cells

brain-specific, endothelial glucose- l-transporter

endoglin

VEGF-receptor-1 (flt-1)

VEGF-receptor-2 (flk-1, KDR)

VEGF-receptor-3 (flt-4)

tie-1 or tie-2

B61-receptor (Eck receptor)

B61

endothelin, specifically endothelin B or endothelin-1

endothelin receptors, in particular the endothelin B receptor

mannose-6-phosphate receptors

von Willebrand factor

PECAM-1

ICAM-3

IL-1α, IL-1β

IL-1 receptor

vascular cell adhesion molecule (VCAM-1)

synthetic activator sequences

Synthetic activator sequences composed of oligomerized binding sites for transcription factors which are preferentially or selectively active in endothelial cells may also be used as alternative to natural endothelial-cell-specific promoters. An example is the transcription factor GATA-2, whose binding site is in the endothelin-1 gene 5′-TTATCT-3′[Lee et al., Biol. Chem. 266, 16188 (1991), Dormann et al., J. Biol. Chem. 267, 1279 (1992) and Wilson et al., Mol. Cell Biol. 10, 4854 (1990)].

f2) Promoters or activator sequences activated in cells in the vicinity of activated endothelial cells

VEGF

The gene-regulatory sequences for the VEGF gene are the 5′-flanking region, the 3′-flanking region, the c-Src gene or the v-Src gene

steroid hormone receptors and their promoter elements (Truss and Beato, Endocr. Rev. 14, 459 (1993)), in particular the mouse mammary tumor virus promoter

f3) Promoter or activator sequences activated in muscle cells, in particular smooth muscle cells

tropomyosin

α-actin

α-myosin

PDGF receptor

FGF receptor

MRF-4

phosphofructokinase A

phosphoglycerate mutase

troponin C

myogenene

endothelin A receptors

desmin

VEGF

The gene-regulatory sequences for the VEGF gene have already been given in the section “Promoters activated in cells in the vicinity of activated endothelial cells” (see above)

“artificial” promoters

Factors of the helix-loop-helix (HLH) family (MyoD, Myf-5, myogenene, MRF4) are described as muscle-specific transcription factors. The muscle-specific transcription factors furthermore include zinc finger protein GATA-4.

The HLH proteins and GATA-4 show muscle-specific transcription not only with promoters of muscle-specific genes, but also in the heterologous context, thus also with artificial promoters. Such artificial promoters are, for example, multiple copies of the (DNA) binding site for muscle-specific HLH proteins, such as the E box (Myo D) (for example 4× AGCAGGTGTTGGGAGGC, SEQ ID NO: 2); or multiple copies of the DNA binding site for GATA-4 of the α-myosin heavy chain gene (for example 5′-GGCCGATGGGCAGATAGAGGGGGCCGATGGGCAGA-TAGAGG3′, SEQ ID NO: 3)

f4) Promoters and activator sequences, activated in glia cells

These include, in particular, the gene-regulatory sequences or elements of genes which encode, for example, the following proteins:

the Schwann-cell-specific protein periaxin

glutamine synthetase

the glia-cell-specific protein (glial fibrillary acid protein =GFAP)

the glia-cell protein S100b

IL-6 (CNTF)

5-HT receptors

TNFα

IL-10

insulin-like growth factor receptor I and II

VEGF

The gene-regulatory sequences for the VEGF gene have already been given above.

f5) Promoters and activator sequences which are activated in hematopoietic cells.

Such gene-regulatory sequences include promoter sequences for genes for a cytokine or its receptor which are expressed in hematopoietic cells or in neighboring cells such as, for example, the stroma.

These include promoter sequences for, for example, the following cytokines and their receptors:

stem cell factor receptor

stem cell factor

IL-1α

IL-1 receptor

IL-3

IL-3 receptor (α-subunit)

IL-3 receptor (β-subunit)

IL-6

IL-6 receptor

GM-CSF

GM-CSF receptor (α-chain)

interferon regulatory factor 1 (IRF-1)

The promoter of IRF-1 is activated equally well by IL-6 and by IFNγ or IFNβ

erythropoietin

erythropoietin receptor.

f6) Promoters and activator sequences which are activated in lymphocytes and/or macrophages

These include, for example, the promoter and activator sequences of the genes for cytokines, cytokine receptors and adhesion molecules and receptors for the Fc fragment of antibodies.

These include, for example,

IL-1 receptor

IL-1α

IL-LB

IL-2

IL-2 receptor

IL-3

IL-3 receptor (α-subunit)

IL-3 receptor (β-subunit)

IL-4

IL-4 receptor

IL-5

IL-6

IL-6 receptor

interferon regulatory factor 1 (IRF-1)

(The promoter of IRF-1 is activated equally well by IL-6 as by IFNγ or IFNβ).

IFNγ responsive promoter

IL-7

IL-8

IL-10

IL-11

IFNγ

GM-CSF

GM-CSF receptor (α-chain)

IL-13

LIF

macrophage colony stimulating factor (M-CSF) receptor

type I and II macrophage scavenger receptors

MAC-1 (leukocyte function antigen)

LFA-1α (leukocyte function antigen)

p150,95 (leukocyte function antigen)

f7) Promoter and activator sequences which are activated in synovial cells

These include the promoter sequences for matrix metalloproteinases (MMP), for example for:

MMP-1 (interstitial collagenase)

MMP-3 (stromelysin/transin)

These include, furthermore, the promoter sequences for tissue inhibitors of metalloproteinases (TIMP), for example

TIMP-1

TIMP-2

TIMP-3

f8) Promoters and activator sequences which are activated in leukemia cells

These include, for example, promoters for

c-myc

HSP-70

bcl-1/cyclin D-1

bcl-2

IL-6

IL-10

TNFα, TNFβ

HOX-11

BCR-Ab1

E2A-PBX-1

PML-RARA (promyelocytic leukemia—retinoic acid receptor)

c-myc

c-myc proteins bind to, and activate, multimers of the nucleotide sequence

(5′-GGAAGCAGACCAGCTGGTCTG CTTCC-3′, SEQ ID NO: 1) which is termed Myc E box

f9) Promoters or activator sequences which are activated in tumor cells

A gene-regulatory nucleotide sequence, with which transcription factors which are either formed or active in tumor cells interact, is envisaged as promoter or activator sequence.

Preferred promoters or activator sequences for the purposes of the present invention include gene-regulatory sequences or elements of genes which encode proteins formed, in particular, in cancer cells or sarcoma cells. Thus, the promoter of the N-CAM protein is preferably used in the case of small-cell bronchial carcinomas, the protomer of the hepatitis growth factor receptor or of L-plastin in the case of ovarian carcinomas, and the promoter of L-plastin or of polymorphic epithelial mucin (PEM) is preferably used in the case of pancreatic carcinomas.

II) The Recombinant Transactivator [Component b)]

In the simplest case, the recombinant transactivator consists of a DNA-binding domain [component b1)] and a transactivation domain which is high in glutamine, Ser and/or Thr [component b2)].

In the case of a pharmacologically controllable recombinant transactivator [component b′)], components b3) and b4) for the coupling substance-binding proteins are introduced, in the case of an oncogen- or virus-controlled recombinant transactivator [component b″)], component b5) for the binding protein for a regulatory protein is introduced.

The operativeness of component b), b′) or b″) can be increased by introducing a nuclear localization signal (NLS). The NLS of SV40 (Dingwall et al., TIBS 16, 478 (1991)) is an example of an NLS which may be used.

1) The DNA-binding domain [component b1)]

The DNA-binding domain represents at least one sequence

of the cDNA for the DNA-binding domain of the Gal4 protein (amino acids 1 to 147; Chasman und Kornberg, Mol. Cell Biol. 10, 2916 (1990)) or

of the LexA protein (amino acids 1 to 81; Kim et al., Science 255, 203 (1992) or the entire LexA protein (amino acids 1 to 202; Brent et al., Cell 43, 729 (1985)) or

of the lac repressor (lac I) protein (Brown et al., Cell 49, 603 (1987); Fuerst et al., PNAS USA 86, 2549 (1989)) or

of the tetracycline repressor(tet-R) proteins (Gossen et al., PNAS USA 89, 5547 (1992); Dingermann et al., EMBO J. 11, 1487 (1992)) or

of the ZFHD1 protein (Pomerantz et al., Science 267, 93 (1995)).

2) The Transactivation Domain [Component b2)]

The DNA to be used for the purposes of the invention is of those transactivation domains which are high in glutamine, serine and/or threonine.

The term high means for the purposes of the present invention that the transactivation domain comprises a total of

at least 20× glutamine (at least 20× glutamine means at least 20 glutamine residues)

at least 10× serine and/or (at least 10× serine means at least 10 serine residues)

at least 10× threonine (at least 10× thereonine means at least 10 thereonine residues)

These transactivation domains include, for the purposes of the invention, for example the

activation domain of Oct-2 (amino acids 438 to 479; Tanaka et al., Mol. Cell Biol. 14, 6064 (1994)) or amino acids 3 to 154; Das et al., Nature 374, 657 (1995)) or

activation domain of SP1 (amino acids 340 to 485; Courey and Tijan, Cell 55, 887 (1988)) or

activation domain of NFY-1A (amino acids 1 to 132 or 1 to 233; Li et al., J. Biol. Chem. 267, 8984 (1992); van Hujisduijnen et al., EMBO J. 9, 3119 (1990); Sinha et al., J. Biol. Chem. 92, 1624 (1995); Coustry et al., J. Biol. Chem. 270, 468 (1995)) or

3) The Coupling-substance [Component f)]-binding Proteins A [Component b3)] and B [Component b4)]

Examples of coupling substances and the corresponding proteins A and B have already been described in detail in Patent Application DE19651443.6, which are incorporated by reference.

These proteins A and B include, for example:

for the coupling substance: rapamycin or rapamycin analogs

the FK506-binding protein (FKBP)

the FKBP-rapamycin-associated protein which binds to the rapamycin FKBP complex, or its sub-sequence which binds to the rapamycin-FKBP complex (FRAP)

instead of using genes for FKBP and FRAP, it is possible to use genes for rec. Fv which bind to rapamycin and/or inhibit the binding of FKBP, or of FRAP, to rapamycin

for the coupling substance: dimers (FK1012) of FK506

the FK506-binding protein (FKBP)

calcineurin or its sub-sequence which binds to the FK506 complex

instead of the gene for calcineurin, it is possible to insert the gene for a rec. Fv which inhibits the binding of FK506 to calcineurin

for the coupling substance dimers of cyclosporin A

cyclophilin

calcineurin or its sub-sequence which binds to the cyclosporin A/cyclophilin complex

instead of the gene for cyclophilin, it is possible to introduce the gene for a rec. Fv which inhibits the binding of cyclosporin A to cyclophilin

for the coupling substance: monomers of cyclosporin A with the following binding proteins

cyclophilin

gene for a rec. Fv which binds to cyclosporin A in the cyclophilin/cyclosporin A complex

as an alternative to cyclophilin, it is possible to use genes for different rec. Fvs which bind to different epitopes of cyclosporin A

for the coupling substance: methotrexate

antibodies or antibody fragments (rec. Fv) against methotrexate

antibodies or antibody fragments (rec. Fv) against the pteridine group

antibodies or antibody fragments (rec. Fv) against the benzene group

dihydrofolate reductase

for the coupling substance: gentamycin

antibodies or antibody fragments (rec. Fv) against gentamycin

for the coupling substance:

antibodies or antibody fragments (rec. Fv) against

for the coupling substance: cephalexin

antibodies or antibody fragments (rec. Fv) against the acyl side chain in the C-7 position of cephem

for the coupling substance: folic acid

folic acid-binding protein

antibodies or antibody fragments (rec. Fv) against folic acid

for the coupling substance: retinoic acid

retinoic-acid-binding domain of the cellular retinoic-acid-binding protein

antibodies or antibody fragments (rec. Fv) against retinoic acid

for the coupling substance:

antibodies or antibody fragments (rec. Fv) against amoxicillin

antibodies or antibody fragments (rec. Fv) against the benzylpenicilloyl group

antibodies or antibody fragments (rec. Fv) against penicillin

the penicillin-binding protein

for the coupling substance: 4-hydroxy-tamoxifen or tamoxifen

estrogen-binding domain of the estrogen receptor protein

antibodies or antibody fragments (rec. Fv) against the estrogen receptor estrogen or 4-hydroxy-tamoxifen complex

for the coupling snbstance: tetracycline

the tetracycline repressor protein

antibodies and antibody fragments against tetracycline

for the coupling substance: conjugate of tetracycline and isopropyl-β-D-thiogalactoside

the tetracycline repressor protein

the lac repressor (lac I) protein

4) The Binding Protein for a Regulatory Protein [Component b5)]

A large number of cellular binding proteins for regulatory proteins have already been described [Zwicker and Müller, Progress in Cell Cycle Res. 1: 91 (1995); Boulikas et al., Int. J. Oncol. 6: 271 (1995); Pawson, Nature 373: 573 (1995); Cotter, Leuk. Lymph. 18: 231 (1995); Hesketh, the Oncogene Facts Book Acad. Press, ISBN 0-12-344550-7 (1995); Miller and Sarver, Nature Med. 3: 389 (1997)].

Suitable for the purposes of the invention are, in particular, binding proteins or their binding sequences for those regulatory proteins which are only weakly expressed in diseased cells, whose binding to the binding sequence is inhibited, which are not present in free form, or only in small amounts, due to an excess of the binding sequence, or whose function is otherwise adversely affected or altered, for example by mutation.

Such regulator proteins include, for example, the proteins which are expressed by tumor suppressor genes.

A choice of such regulatory proteins and their corresponding binding proteins and their binding sequences is given in the examples which follow, and does not constitute a limitation of the invention:

| | Component b5) (cellular binding |
|---|---|
| | protein with binding |
| Regulatory protein | sequence for the regulatory protein) |
| p53 | MDM-2 |
| PRb | transcription factor E2F, −1, −2, −3 |
| | cyclin-D1, -D2, -D3, or -C |
| | cyclin-A, -E |
| | transcription factor PU.1 |
| | transcription factor Elf-1 |
| p130 | transcription factor E2F-5 |
| | cyclin A, -E |
| Max | Myc |
| MAD | Myc |
| VHL | elongin C, -B |
| cdk4 | p14, p15, p16, p18, p27, p57, p21 |
| MTS-1 (p16) | cdk4 |
| WT-1 | P53 |
| SMAD2 (MADR2) | DPC4 |
| DPC-4 | SMAD2 |
| β-catenin | LEF-1 |
| LEF-1 | β-catenin |

In a particular embodiment of the present invention, component b5) is a binding sequence of a cell-foreign binding protein for a regulatory protein. Such a cell-foreign binding sequence may be, for example, of viral, bacterial or parasitic origin.

The use of such a cell-foreign binding sequence allows the function of component b) to be inhibited in normal cells since the corresponding regulatory protein is bound to component b5). In infected cells, however, the corresponding regulatory protein is largely bound due to the intracellular production, by the particular pathogen, of the binding protein which contains the binding sequence. Thus, component b) is free and operational in these cells.

In a further particular embodiment of the present invention, component b5) is an antibody or part, i.e., a fragment thereof, of an antibody with binding sequences (VH and VL) for a regulatory protein.

A selection of cell-foreign binding sequences which does not limit the invention is listed in the examples which follow:

| | |
|---|---|
| Regulatory protein | Component b5) |
| | (viral binding protein with binding sequence for the |
| | regulatory protein) |
| p53 | IE 84 of CMV |
| | E1B (55 Kd) of AV |
| | EBNA-5 of EBV |
| | BHFR1 of EBV |
| | E6 of HPV, e.g of HPV-16 or -18 |
| | HBX protein of HBV |
| | T antigen of SV40 |
| PRb | E1A of AV |
| | EBNA-2 of EBV |
| | EBNA-1 or -5 of EBV |
| | E7 of HPV |
| | T antigen of SV40 |
| p130 | E1A of AV |
| CBF-1 (RBP-JK) | EBNA-2 of EBV |
| NF-Kappa B | Tax of HIV |
| Lyn-tyrosinkinase | LMP-1 of EBV |
| | LMP-2A or LMP-2B of EBV |
| Bak | E1B (16 Kd) of AV |
| Bax | E1B (19 kD) of Av |
| Regulatory protein | Component b5) |
| | (viral binding protein with binding sequence for the |
| | regulatory protein) |
| Regulatory protein | Antibody or antibody fragments with binding |
| | sequence (VH, VL) for the regulatory protein |
| P53 | Monoclonalantibodies which are specific for the |
| | unmutated DNA binding domain |
| pRb | Monoclonal antibodies which are specific for active |
| | (unphosphorylated) pRb |
| myc | Monoclonal antibodies specific for the DNA binding |
| | domains |

When selecting an antibody, it is preferred to employ the epitope-binding parts of the antibody FVL and FVH as component b5), which, if the antibody is of murine origin, are in humanized form. Humanization is effected in the manner described by Winter et al. (Nature 349, 293 (1991) and Hoogenbooms et al. (Rev. Tr. Transfus. Hemobiol. 36, 19 (1993)). The antibody fragments are prepared in accordance with the state of the art, for example in the manner described by Winter et al., Nature 349, 293 (1991), Hoogenboom et al., Rev. Tr. Transfus. Hemobiol. 36, 19 (1993), Girol. Mol. Immunol. 28, 1379 (1991) or Huston et al., Int. Rev. Immunol. 10, 195 (1993).

Recombinant antibody fragments are prepared directly from existing hybridomas or are isolated (Winter et al., Annu. Rev. Immunol. 12, 433 (1994)) from libraries of murine or human antibody fragments with the aid of phage-display technology (Smith, Science 228, 1315, (1985)). These antibody fragments are then employed directly at the genetic level for the coupling to components b1) and b2).

To prepare recombinant antibody fragments from hybridomas, the genetic information which encodes the antigen-binding domains (VH, VL) of the antibodies is obtained by isolating the mRNA, reverse-transcribing the RNA into cDNA and subsequently amplifying by means of polymerase chain reaction (Saiki et al., Science 230, 1350 (1985)) and using oligonucleotides which are complementary to the 5′ and 3′ ends, respectively, of the variable fragments (Orlandi et al. 1989). The VH and VL fragments are then cloned into bacterial expression vectors, for example in the form of Fv fragments (Skerra and Pluckthun, Science 240, 1038 (1988)), single-chain Fv fragments (scFv) (Bird et al., Science 242, 423 (1988); Huston et al., PNAS USA 85, 5879 (1988)) or as Fab fragments (Better et al., Science 240, 1041 (1988)).

New antibody fragments may also be isolated directly from antibody libraries (immune libraries, naive libraries) of murine or human origin by means of phage-display technology (McCafferty et al., Nature 348, 552 (1990); Reitling et al., Gene 104, 147 (1991); McCafferty et al., Nature 348, 552 (1990); Hoogenboom et al., Nucl. Acid Res. 19, 4133 (1991); Barbas et al., PNAS USA 88, 7978 (1991); Marks et al., J. Mol. Biol. 222, 581 (1991); Hawkins et al., J. Mol. Biol. 226, 889 (1992); Marks et al., Bio/Technol. 11, 1145 (1993)).

Immune libraries are prepared by PCR amplification of the variable antibody fragments from B lymphocytes of immunized animals (Sastry et al., PNAS USA 86, 5728 (1989); Ward et al., Nature 341, 544 (1989); Clackson et al., Nature 352, 624 (1991)) or patients (Mullinax et al., PNAS USA 87, 8095 (1990); Barbas et al., PNAS USA 88, 7978 (1991)).

The affinity of antibody fragments can be improved further by means of phage-display technology, novel libraries of existing antibody fragments being prepared by random (Hawkins et al., J. Mol. Biol. 226, 889 (1992); Gram et al., PNAS USA 89, 3576 (1992)), codon-based (Glaser et al., J. Immunol. 149, 3903 (1992)) or directed mutagenesis (Balint and Larrick, Gene 137, 109 (1993)), by shuffling the chains of individual domains with fragments from naive repertoires (Marks et al., Bio/Technol. 10, 779 (1992)) or with the aid of bacterial mutator strains (Low et al., J. Mol. Biol. 260, 359 (1996)) and antibody fragments having improved properties being isolated by reselection under stringent conditions (Hawkins et al., J. Mol. Biol. 226, 889 (1992)).

III) The DNA-binding Sequence for Component b) [Component c)]

The selection of the DNA-binding sequence depends on the choice of the DNA-binding domains. For example, the following possibilities exist for the examples of the DNA-binding domains which are given under 11.1):

at least one binding sequence for the Gal4 protein [nucleotide sequence: 5′-CGGACAACTGTTGACCG-3′, SEQ ID NO: 4]; Chasman and Kornberg, Mol. Cell Biol. 10, 2916 (1990) or [nucleotide sequence: 5′-CGGAGGACTGTCCTCCG 3′, SEQ ID NO: 5]; or [nucleotide sequence: 5′-CGGAGTACTGTCCTCCG-3′, SEQ ID NO: 6]; Giniger et al., PNAS USA 85, 382 (1988)

at least one binding sequence [nucleotide sequence: 5′-TACTGTATGTACATA-CAGTA-3′, SEQ ID NO: 7]; for the LexA protein [LexA operator; Brent et al., Nature 612, 312 (1984)]

at least one Lac operator binding sequence (nucleotide sequence: 5′-GAATTGTG AGGCTCACAATTC-3′, SEQ ID NO: 8); for the lac I repressor protein (Fuerst et al., PNAS USA 86, 2549 (1989); Simons et al., PNAS USA 81, 1624 (1984))

at least one tetracycline operator(tet 0) binding sequence (nucleotide sequence: 5′-TCGAGTTTACCACTCCCTATCAGTGATAGAGAAAAGTGAAAG-3′, SEQ ID NO: 9); for the tetracycline repressor (tet R) protein

at least one binding sequence (nucleotide sequence: 5′-TAATGATGGGCG-3′, SEQ ID NO: 10); for the ZFHD-1 protein (Pomeranth et al., Science 267, 93 (1995))

IV) The Minimal Promoter Containing CDE-CHR or E2FBS-CHR [Component d)]

Examples of fragments which can be used are those

of the cdc25C gene (nucleic acids −20 to +121 or nucleic acids −20 to +50) of the cdc2 (cdk-1) gene (nucleic acids −26 to +121; Liu et al., Nucl. Acids Res. 24, 2905 (1996))

of the cyclinA gene (nucleic acids −40 to +94; Liu et al., Nucl. Acids Res. 24, 2905 (1996))

of the B-myb gene (nucleic acids −50 to +50; Liu et al., Nucl. Acids Res. 24, 2905 (1996))

V) Effector Genes [Component e)]

For the purposes of the invention, the effector genes encode an active compound for the prophylaxis and/or therapy of a disease. Effector genes and promoter sequences are to be selected with regard to the nature of the therapy of the disease and taking into consideration the target cell to be transduced.

For example, the following combinations of promoter sequences (examples see section C I) and effector genes are to be selected in the case of the following diseases (a detailed description has already been given in the Patent Applications EP-A 0 777 739, EP-A 0 777 740, EP-A 0 804 601, EP-A 0 807 183, EP-A 0 790 313 EP-A 0 805 209 and EP-A 0 848 063 which are incorporated by reference).

a) Tumor Therapy

a.1) Target cells:

proliferating endothelial cells or

stroma cells and muscle cells which are adjacent to the endothelial cell, or

tumor cells or leukemia cells

a.2) Promoters:

endothelial-cell-specific and cell-cycle-specific, or

cell-nonspecific or muscle-cell-specific and cell-cycle-specific, or

tumor-cell-specific (solid tumors, leukemias) and cell-cycle-specific

a.3) Effector genes for cell proliferation inhibitors, for example for

the retinoblastom a protein (pRb=p110) or the related p107 and p130 proteins

The retinoblastoma protein (pRb/p110) and the related p107 and p130 proteins are inactivated by phosphorylation. Genes of these cell cycle inhibitors which are preferably to be used are those which exhibit mutations for the inactivation sites of the expressed proteins without the function of the latter thereby being adversely affected. Examples of such mutations have been described for the p110.

The DNA sequence for the p107 protein or the p130 protein is mutated analogously.

the p53 protein

The protein p53 is inactivated in the cell either by binding to specific proteins such as MDM2 or by oligomerization of the p53 via the dephosphorylated C-terminal serine. Thus, a preferred DNA sequence for a p53 protein is one which is truncated C-terminally by the serine 392.

the p21 (WAF-1)

the p16 protein

other cdk inhibitors

the GADD45 protein

the bak protein

a.4) Effector genes for coagulation-inducing factors and angiogenesis inhibitors, for example:

plasminogen activator inhibitor-1 (PAI-1)

PAI-2

PAI-3

angiostatin and/or endostatin

interferons (IFNα, IFNβ or IFNγ)

platelet factor 4

IL-12

TIMP-1

TIMP-2

TIMP-3

leukemia inhibitory factor (LIF)

tissue factor (TF) and its coagulation-active fragments

a.5) Effector genes for cytostatic and cytotoxic proteins, for example for

perforin

granzyme

IL-2

IL-4

IL-12

interferons such as, for example IFN-α, IFNβ or IFNγ

TNF, such as TNFα or TNFβ

oncostatin M

sphingomyelinase

magainin and magainin derivatives

a.6) Effector genes for cytostatic or cytotoxic antibodies and for fusion proteins formed between antigen-binding antibody fragments and cytostatic, cytotoxic or inflammatory proteins or enzymes.

The cytostatic or cytotoxic antibodies include those which are directed against membrane structures of endothelial cells as have been described, for example, by Burrows et al. (Pharmac. Ther. 64, 155 (1994)), Hughes et al., (Cancer Res. 49, 6214 (1989)) and Maruyama et al., (PNAS USA 87, 5744 (1990)). They include, in particular, antibodies against the VEGF receptors.

Furthermore, they include cytostatic or cytotoxic antibodies which are directed against membrane structures on tumor cells. Such antibodies were reviewed, for example, by Sedlacek et al., Contrib. to Oncol. 32, Karger Verlag, Munich (1988) and Contrib. to Oncol. 43, Karger Verlag, Munich (1992). Other examples are antibodies against sialyl Lewis; against peptides on tumors which are recognized by T lymphocytes; against oncogen-expressed proteins; against gangliosides such as GD3, GD2, GM2, 9-0-acetyl GD3, fucosyl GM1; against blood group antigens and their precursors; against antigens on the polymorphic epithelial mucin; against antigens on heat shock proteins

They furthermore include antibodies which are directed against membrane structures of leukemia cells. A large number of such monoclonal antibodies have already been described for diagnostic and therapeutic methods (reviews in Kristensen, Danish Medical Bulletin 41, 52 (1994); Schranz, Therapia Hungarica 38, 3 (1990); Drexler et al., Leuk. Res. 10, 279 (1986); Naeim, Dis. Markers 7, 1 (1989); Stickney et al., Curr. Opin. Oncol. 4, 847 (1992); Drexler et al., Blut 57, 327 (1988); Freedman et al., Cancer Invest. 9, 69 (1991)). Depending on the type of leukemia, suitable ligands are, for example, monoclonal antibodies or their antigen-binding antibody fragments which are directed against the following membrane antigens:

| Cells | Membrane antigen |
|---|---|
| AML | CD13 |
| | CD15 |
| | CD33 |
| | CAMAL |
| | sialosyl-Le |
| B-CLL | CD5 |
| | CD1c |
| | CD23 |
| | idiotypes and isotypes of the membrane |
| | immunoglobulins |
| T-CLL | CD33 |
| | M38 |
| | IL-2 receptors |
| | T-cell receptors |
| ALL | CALLA |
| | CD19 |
| | non-Hodgkin's lymphoma |

The humanization of murine antibodies, the preparation and the optimization of the genes for Fab and rec. Fv fragments are performed in accordance with the technique known to the skilled worker (Winter et al., Nature 349, 293 (1991); Hoogenbooms et al., Rev. Tr. Transfus. Hemobiol. 36, 19 (1993); Girol. Mol. Immunol. 28, 1379 (1991) or Huston et al., Intern. Rev. Immunol. 10, 195 (1993)). Fusion of the rec. Fv fragments with genes for cytostatic, cytotoxic or inflammatory proteins or enzymes is also performed in accordance with the state of the art known to the skilled worker.

a.7) Effector genes for fusion proteins formed between target-cell-binding ligands and cytostatic and cytotoxic proteins. The ligands include all substances which bind to membrane structures or membrane receptors on endothelial cells. Examples of these include

Cytokines such as, for example, IL-1 or growth factors or their fragments or sub-sequences thereof which bind to receptors which are expressed by endothelial cells, such as, for example, PDGF, bFGF, VEGF, TGF.

They furthermore include adhesion molecules which bind to activated and/or proliferating endothelial cells. These include, for example, SLex, LFA-1, MAC-1, LECAM-1, VLA-4 or vitronectin.

They furthermore include substances which bind to membrane structures or membrane receptors of tumor or leukemia cells. These include, for example, growth factors or their fragments or sub-sequences of these which bind to receptors which are expressed by leukemia cells or tumor cells.

Such growth factors have already been desribed (reviews in Cross et al., Cell 64, 271 (1991), Aulitzky et al., Drugs 48, 667 (1994), Moore, Clin. Cancer Res. 1, 3 (1995), Van Kooten et al., Leuk. Lymph. 12, 27 (1993)).

The fusion of the genes for these ligands which bind to the target cell and cytostatic, cytotoxic or inflammatory proteins or enzymes is carried out in accordance with the state of the art using the methods known to the skilled worker.

a.8) Effector genes for inflammation inducers, for example for

IL-1

IL-2

RANTES (MCP-2)

monocyte chemotactic and activating factor (MCAF)

IL-8

macrophage inflammatory protein-1 (MIP-1α, -β)

neutrophil activating protein-2 (NAP-2)

IL-3

IL-5

human leukemia inhibitory factor (LIF)

IL-7

IL-11

IL-13

GM-CSF

G-CSF

M-CSF

cobra venom factor (CVF) or sub-sequences of CVF which correspond operatively to human complement factor C3b, i.e. which are capable of binding to complement factor B and which, after cleavage by factor D, constitute a C3 convertase

human complement factor C3 or its sub-sequence C3b

cleavage products of human complement factor C3 which resemble CVF operatively and structurally

bacterial proteins which activate complement or trigger inflammations, such as porins of Salmonella typhi murium, clumping factors of Staphylococcus aureus, modulins, in particular from Gram-negative bacteria, major outer membrane protein of legionellas or of Haemophilus influenza type B or of klebsiellas, or M molecules from group G streptococci.

a.9) Effector genes for enzymes for the activation of precursors of cytostatic agents, for example for enzymes which cleave inactive precursors (prodrugs) into active cytostatic agents (drugs).

Such substances, and the corresponding prodrugs and drugs, have already been reviewed by Deonarain et al. (Br. J. Cancer 70, 786 (1994)), Mullen, Pharmac. Ther. 63, 199 (1994)) and Harris et al. (Gene Ther. 1, 170 (1994)). For example, use is to be made of the DNA sequence for one of the following enzymes:

herpes simplex virus thymidine kinase

varicella zoster virus thymidine kinase

bacterial nitroreductase

bacterial β-glucuronidase

plant β-glucuronidase from Secale cereale

human β-glucuronidase

human carboxypeptidase (CB), for example mast cell CB-A, pancreatic CB-B or bacterial carboxypeptidase

bacterial β-lactamase

bacterial cytosin deaminase

human catalase or peroxidase

phosphatase, in particular human alkaline phosphatase, human acid prostate phosphatase or type 5 acid phosphatase

oxidase, in particular human lysyl oxidase or human acid D-aminooxidase

peroxidase, in particular human glutathione peroxidase, human eosinophilic peroxidase or human thyroid peroxidase

galactosidase

b) Therapy of Autoimmune Diseases and Inflammations

(a detailed description has already been given in Patent Application EP-A 0 807 183, which is incorporated by reference)

b. 1) Target cells:

proliferating endothelial cells or

macrophages and/or lymphocytes or

synovial cells

b.2) Promoters:

endothelial-cell-specific and cell-cycle-specific or

macrophage- and/or lymphocyte-specific and/or cell-cycle-specific or

synovial-cell-specific and/or cell-cycle-specific

b.3) Effector genes for the therapy of allergies, for example for

IFNβ

IFNγ

IL-10

antibodies or antibody fragments against IL-4

soluble IL-4 receptors

IL-12

TGFβ

b.4) Effector genes for preventing the rejection of transplanted organs, for example for

L-10

TGFβ

soluble IL-1 receptors

soluble IL-2 receptors

IL-1 receptor antagonists

soluble IL-6 receptors

immunosuppressive antibodies or their VH- and VL-containing fragments, or their VH and VL fragments which are bonded via a linker.

Immunosuppressive antibodies are, for example, antibodies which are specific for the T-cell receptor or its CD3 complex, or antibodies against CD4 or CD8, furthermore against the IL-2 receptor, the IL-1 receptor or the IL-4 receptor, or against the adhesion molecules CD2, LFA-1, CD28 or CD40

b.5) Effector genes for the therapy of antibody-mediated autoimmune diseases, for example for

TGFβ

IFNα

IFNβ

IFNγ

IL-12

soluble IL-4 receptors

soluble IL-6 receptors

immunosuppressive antibodies or their VH- and VL-containing fragments

b.6) Effector genes for the therapy of cell-mediated autoimmune diseases, for example for

IL-6

IL-9

IL-10

IL-13

TNFα or TNFβ

IL-13

an immunosuppressive antibody or its VH- and VL-containing fragments

b.7) Effector genes for inhibitors of cell proliferation, cytostatic or cytotoxic proteins and enzymes for the activation of precursors of cytostatic agents

Examples of genes which encode such proteins have already been mentioned in the section “Effector genes for the therapy of tumors”.

In the same form as already described above, use can be made for the purposes of the invention of structural genes which encode fusion proteins formed from antibodies or Fab or rec. Fv fragments of these antibodies, or other ligands which are specific for the target cell, and the abovementioned cytokines, growth factors, receptors, cytostatic or cytotoxic proteins and enzymes.

b.8) Effector genes for the therapy of arthritis

For the purposes of the invention, structural genes are selected whose expressed protein directly or indirectly inhibits the inflammation in, for example, the joint and/or promotes the reconstitution of extracellular matrix (cartilage, connective tissue) in the joint.

These include, for example,

IL-1 receptor antagonist (IL-1-RA);

IL-1-RA inhibits the formation of IL-la, B

soluble IL-1 receptor;

soluble IL-1 receptor binds and inactivates IL-1

IL-6

IL-6 increases the secretion of TIMP and superoxides and decreases the secretion of IL-1 and TNFα by synovial cells and chondrocytes

soluble TNF receptor

soluble TNF receptor binds and inactivates TNF.

IL-4

IL-4 inhibits the formation and secretion of IL-1, TNFα and MMP

IL-10

IL-10 inhibits the formation and secretion of IL-1, TNF∝ and MMP and increases the secretion of TIMP

insulin-like growth factor (IGF-1)

IGF-1 stimulates the synthesis of extracellular matrix.

TGFβ, specifically TGFB1 and TGFB2

TGFβ stimulates the synthesis of extracellular matrix.

superoxide dismutase

TIMP, specifically TIMP-1, TIMP-2 or TIMP-3

c) Therapy of the Deficient Formation of Blood Cells

(a detailed description has already been given in Patent Application EP-A 0 807 183, which is incorporated by reference)

c.1) Target cells:

proliferating, immature cells of the hematopoietic system or

stroma cells which are adjacent to the hematopoietic cells

c.2) Promoters:

specific for hematopoietic cells and/or cell-cycle-specific

cell-nonspecific and cell-cycle-specific

c.3) Effector genes for the therapy of anemia, for example for

erythropoietin

c.4) Effector genes for the therapy of leukopenia, for example for

G-CSF

GM-CSF

M-CSF

c.5) Effector genes for therapy of thrombocytopenia, for example for

IL-3

leukemia inhibitory factor (LIF)

IL-11

thrombopoietin

d) Therapy of Damage to the Nervous System

(a detailed description has already been given in Patent Application EP-A 0 807 183, which is incorporated by reference)

d. 1) Target cells:

glia cells or

proliferating endothelial cells

d.2) Promoters:

glia cell-specific and cell-cycle-specific or

endothelial-cell-specific and cell-cycle-specific or

nonspecific and cell-cycle-specific

d.3) Effector genes for neuronal growth factors, for example

FGF

nerve growth factor (NGF)

brain-derived neurotrophic factor (BDNF)

neurotrophin-3 (NT-3)

neurotrophin-4 (NT-4)

ciliary neurotrophic factor (CNTF)

d.4) Effector genes for enzymes, for example for - tyrosine hydroxylase

dopade carboxylase

d.5) Effector genes for cytokines and their inhibitors which inhibit or neutralize the neurotoxic effect of TNFα, for example for

TGFβ

soluble TNF receptors

TNF receptors neutralize TNFα

IL-10

IL-10 inhibits the formation of IFNγ, TNFα, IL-2 and IL-4

soluble IL-1 receptors

IL-1 receptor I

IL-1 receptor II

soluble IL-1 receptors neutralize the activity of IL-1

IL-1 receptor antagonist

soluble IL-6 receptors

e) Therapy of Disturbances of the Blood Coagulation System and the Blood Circulation System

(a detailed description has already been given in Patent Applications EP-A 0 777 739, EP-A 0 790 313, EP-A 0 805 209 and EP-A 0 848 063, which are incorporated by reference)

e. 1) Target cells:

endothelial cells or

proliferating endothelial cells or

somatic cells in the vicinity of endothelial cells and smooth muscle cells or

macrophages

e.2) Promoters:

cell-nonspecific and cell-cycle-specific or

specific for endothelial cells, smooth muscle cells or macrophages and cell-cycle-specific

e.3) Effector genes for inhibiting coagulation, or for promoting fibrinolysis, for example for

tissue plasminogen activator (tPA)

urokinase-type plasminogen activator (uPA)

hybrids of tPA and uPA

protein C

hirudin

serine proteinase inhibitors (serpines), such as, for example, C-1S inhibitor, α1-antitrypsin or antithrombin III

tissue factor pathway inhibitor (TFPI)

e.4) Effector genes for promoting coagulation, for example for

F VIII

FIX

von Willebrand factor

F XIII

PAI-1

PAI-2

tissue factor and fragments thereof

e.5) Effector genes for angiogenesis factors, for example for

VEGF

FGF

e.6) Effector genes for lowering the blood pressure, for example for

kallikrein

endothelial cell nitric oxide synthase

e.7) Effector genes for inhibiting the proliferation of smooth muscle cells after injury to the endothelial layer, for example for

an antiproliferative, cytostatic or cytotoxic protein or

an enzyme for cleaving precursors of cytostatic agents into cytostatic agents as already indicated above (under tumor) or

a fusion protein between one of these active compounds and a ligand, for example an antibody or antibody fragments which are specific for muscle cells

e.8) Effector genes for other blood plasma proteins, for example for

albumin

C1 inactivator

serum cholinesterase

transferrin

1-antritrypsin

f) Inoculations

(a detailed description has already been given in Patent Applications EP-A 0 807 183, EP-A 0 790 313, EP-A 0 860 445, which are incorporated by reference)

f.1) Target cells:

muscle cells or

macrophages and/or lymphocytes

f.2) Promoters:

nonspecific and cell-cycle-specific or

target-cell-speicifc and cell-cycle-specific

f.3) Effector genes for the prophylaxis of infectious diseases

The possibilities of preparing effective vaccines by conventional means are limited.

As a consequence, the technology of DNA vaccines was developed. However, these DNA vaccines raise questions regarding their efficacy (Fynan et al., Int. J.

Immunopharm. 17, 79 (1995); Donnelly et al., Immunol. 2, 20 (1994)).

A better efficacy of the DNA vaccines can be expected in accordance with the present invention.

The active substance to be selected is the DNA of a protein formed by the pathogen which leads, by means of triggering an immune reaction, i.e. by means of antibody binding and/or by means of cytotoxic T-lymphocytes, to the neutralization and/or destruction of the pathogen. Such so-called neutralization antigens are already being applied as vaccination antigens (see review in Ellis, Adv. Exp. Med. Biol. 327, 263 (1992)).

Preferred for the purposes of the invention is the DNA which encodes neutralization antigens of the following pathogens:

influenza A virus

HIV

rabies virus

HSV (herpes simplex virus)

RSV (respiratory syncytial virus)

parainfluenza virus

rotavirus

VZV (varicella zoster virus)

CMV (cytomegalovirus)

measles virus

HPV (human papilloma virus)

HBV (hepatitis B virus)

HCV (hepatitis C virus)

HDV (hepatitis D virus)

HEV (hepatitis E virus)

HAV (hepatitis A virus)

Vibrio cholera antigen

Borrelia burgdorferi

Helicobacter pylori

malaria antigen

However, such active substances also include, for the purposes of the invention, the DNA of an antiidiotype antibody or of its antigen-binding fragments whose antigen binding structures (the complementary determining regions) constitute copies of the protein or carbohydrate structure of the neutralization antigen of the pathogen.

Such antiidiotype antibodies can replace, in particular, carbohydrate antigens in bacterial pathogens.

Such antiidiotypic antibodies and their cleavage products have been reviewed by Hawkins et al. (J. Immunother. 14, 273 (1993)) and Westerink and Apicella (Springer Seminars in Immunopathol. 15, 227 (1993)).

f.4) Effector genes for “tumor vaccines”

These include antigens on tumor cells. Such antigens have been reviewed, for example, by Sedlacek et al., Contrib. to Oncol. 32, Karger Verlag, Munich (1988) and Contrib. to Oncol 43, Karger Verlag, Munich (1992).

Other examples are the genes for the following antigens, or for antiidiotype antibodies which correspond to the following antigens:

sialyl Lewis

peptides on tumors which are recognized by T-lymphocytes

proteins expressed by oncogenes

blood group antigens and their precursors

antigens on polymorphic epithelial mucin

antigens on heat shock proteins

g) The Therapy of Chronic Infectious Diseases

(a detailed description has already been given in Patent Applications EP-A 0 807 183and EP-A 0 860 445, which are incorporated by reference)

g.1) Target cell:

liver cell

lymphocyte and/or macrophage

epithelial cell

endothelial cell

g.2) Promoters:

virus-specific or cell-specific and cell-cycle-specific

g.3) Effector genes, for example for

a protein which exhibits cystatic, apoptotic or cytotoxic effects.

an enzyme which cleaves a precursor of an antiviral or cytotoxic substance into the active substance.

g.4) Effector gene for antiviral proteins

cytokines and growth factors which have an antiviral effect. These include, for example, IFNα, IFNβ, IFN-γ, TNFβ, TNFα, IL-1 oder TGFβ

antibodies of a specificity which inactivates the virus in question, or their VH- and VL-containing fragments, or their VH and VL fragments which are bonded via a linker, prepared as already described.

Examples of antibodies against viral antigen are:

anti-HBV

anti-HCV

anti-HSV

anti-HPV

anti-HIV

anti-EBV

anti-HTLV

anti-Coxackie virus

anti-Hantaan virus

a Rev-binding protein. These proteins bind to the Rev RNA and inhibit Rev-dependent posttranscriptional stages in retrovirus gene expression. Examples of Rev-binding proteins are:

RBP9-27

RBP1-8U

RBP1-8D

pseudogenes of RBP1-8

ribozymes which digest the mRNA of genes for cell cycle control proteins, or the mRNA of viruses. Ribozymes which are catalytic for HIV have been reviewed, for example, by Christoffersen et al., J. Med. Chem. 38, 2033 (1995).

g.5) Effector genes for antibacterial proteins

The antibacterial proteins include, for example, antibodies which neutralize bacterial toxins or which opsonize bacteria. These antibodies include antibodies against

meningococci C or B

E. coli

Borrelia

Pseudomonas

Helicobacter pylori

Staphylococcus aureus

VI) Combination of Identical or Different Effector Genes

(a detailed description has been given in EP-A 0 777 739 and EP-A 0 860 445, which are incorporated by reference)

To express two or more effector genes [for example components e, e′, e″], a further component c) and component d) or, preferably, the cDNA of an internal ribosome entry site (IRES) is intercalated in each case between the effector genes in question as regulatory element.

An IRES allows the expression of two DNA sequences linked to each other via an IRES.

Such IRESs have been described, for example, by Montford and Smith (TIG 11, 179 (1995); Kaufman et al., Nucl. Acids Res. 19, 4485 (1991); Morgan et al., Nucl. Acids Res. 20, 1293 (1992); Dirks et al., Gene 128, 247 (1993); Pelletier and Sonenberg, Nature 334, 320 (1988) and Sugitomo et al., BioTechn. 12, 694 (1994)).

For example, it is possible to use the corresponding DNA sequence of the poliovirus IRES sequence (position <140 to >630 of the 5′ UTR).

For the purposes of the invention, it is preferred to link, via further components c) and d) or via an IRES sequence, effector genes which have an additive effect.

Preferred for the purposes of the invention are combinations of effector genes for example for

a) The therapy of tumors

identical or different, cytostatic, apoptotic, cytotoxic and/or inflammatory proteins or

identical or different enzymes for cleaving the precursor of a cytostatic agent

b) The therapy of autoimmune diseases

different cytokines or receptors which have a synergistic effect for inhibiting cellular and/or humoral immune reaction, or

different or identical TIMPs

c) The therapy of deficient formation of blood cells

different, hierarchically sequential cytokines such as, for example, IL-1, IL-3, IL-6 or GM-CSF and erythropoietin, G-CSF or thrombopoietin

d) The therapy of nerve cell damage

a neuronal growth factor and a cytokine or the inhibitor of a cytokine

e) The therapy of disturbances of the blood coagulation system and the blood circulatory system

an antithrombotic agent and a fibrinolytic agent (for example tPA or uPA) or

a cytostatic, apoptotic or cytotoxic protein and an antithrombotic agent or a fibrinolytic agent

several different, synergistically acting blood coagulation factors, for example F VIII and vWF or F VIII and F IX

f) Vaccinations

an antigen and an immunostimulatory cytokine, such as, for example, IL-1α, IL-1β, IL-2, GM-CSF, IL-3 or IL-4 receptor

different antigens of one pathogen or of different pathogens or

different antigens of one tumor type or of different tumor types

g) Therapy of viral infectious diseases

an antiviral protein and a cytostatic, apoptotic or cytotoxic protein

antibodies against different surface antigens of one virus or of several viruses

h) Therapy of bacterial infectious diseases

antibodies against different surface antigens and/or toxins of a microorganism

VII) Introduction of Signal Sequences and Transmembrane Domains

a) To enhance translation, the nucleotide sequence GCCACC or GCCGCC may be inserted at the 3′ end of the promoter sequence and directly at the 5′ end of the start signal (ATG) of the signal or transmembrane sequence (Kozak, J. Cell Biol. 108, 299 (1989)).

b) To facilitate secretion of the expression product of the effector gene, the homologous signal sequence which may be contained in the DNA sequence of the effector gene can be replaced by a heterologous signal sequence which improves extracellular secretion.

Thus, for example, the signal sequence for immunoglobulin (DNA position <63 to >107; Riechmann et al., Nature 332, 323 (1988)) or the signal sequence for CEA (DNA position <33 to >134; Schrewe et al., Mol. Cell Biol. 10, 2738 (1990); Berling et al., Cancer Res. 50, 6534 (1990)) or the signal sequence of human respiratory syncytial virus glycoprotein (cDNA of amino acids <38 to >50 or 48 to 65; Lichtenstein et al., J. Gen. Virol. 77, 109 (1996)) may be inserted.

c) To anchor the active substance into the cell membrane of the transduced cell which forms the active substance, it is possible to introduce a sequence for a transmembrane domain, either instead of or in addition to the signal sequence.

Thus, for example, the transmembrane sequence of human macrophage-colony-stimulating factor (DNA position <1485 to >1554; Cosman et al., Behring Inst. Mitt. 83, 15 (1988)) or the DNA sequence for the signal and transmembrane regions of human respiratory syncytial virus (RSV) glycoprotein G (amino acids 1 to 63 or their sub-sequences, amino acids 38 to 63; Vijaya et al., Mol. Cell Biol. 8, 1709 (1988); Lichtenstein et al., J. Gen. Virol. 77, 109 (1996)) or the DNA sequence for the signal and transmembrane regions of influenza virus neuraminidase (amino acids 7 to 35 or the sub-sequence amino acids 7 to 27; Brown et al., J Virol. 62, 3824 (1988)) may be inserted between the promoter sequence and the sequence of the effector gene.

d) To anchor the active substance into the cell membrane of the transduced cells which form the active substance, it is also possible, however, to insert the nucleotide sequence for a glycophospholipid anchor.

A glycophospholipid anchor is inserted on the 3′ end of the nucleotide sequence for the structural gene, and this can be done in addition to inserting a signal sequence.

Glycophospholipid anchors have been described, for example, for CEA, for N-CAM and for other membrane proteins, such as, for example, Thy-1 (see review Ferguson et al., Ann. Rev. Biochem. 57, 285 (1988)).

e) A further possibility of anchoring active substances to the cell membrane in accordance with the present invention is the use of a DNA sequence for a ligand/active substance fusion protein. The specificity of the ligand of this fusion protein is directed against a membrane structure on the cell membrane of the selected target cell.

e.1) The ligands which bind to the surface of cells include, for example, antibodies or antibody fragments directed against structures on the surface of, for example,

endothelial cells. These include, in particular, antibodies against the VEGF receptors or against kinin receptors

or of muscle cells, such as antibodies against actin or antibodies against angiotensin II receptors or antibodies against receptors for growth factors such as, for example, against EGF receptors or against PDGF receptors or against FGF receptors or antibodies against endothelin A receptors

the ligands also include antibodies or their fragments which are directed against tumor-specific or tumor-associated antigens on the tumor cell membrane. Such antibodies have already been described.

The murine monoclonal antibodies are preferably to be employed in humanized form. As already described, Fab and rec. Fv fragments and their fusion products are prepared using the technology known to the skilled worker.

e.2) The ligands furthermore include all active substances such as, for example, cytokines or adhesion molecules, growth factors or their fragments or sub-sequences thereof, or mediators or peptide hormones which bind to membrane structures or membrane receptors on the selected cell in question. They include, for example,

ligands for endothelial cells, such as IL-1, PDGF, bFGF, VEGF, TGGB (Pusztain et al., J. Pathol. 169, 191 (1993)) or kinin and derivatives, or kinin analogs.

They furthermore include adhesion molecules. Such adhesion molecules such as, for example, SLex, LFA-1, MAC-1, LeCAM-1, VLA-4 or vitronectin and derivatives or analogs of vitronectin have already been described for endothelial cells (reviews in Augustin-Voss et al., J. Cell Biol. 119, 483 (1992); Pauli et al., Cancer Metast. Rev. 9, 175 (1990); Honn et al., Cancer Metast. Rev. 11, 353 (1992); Varner et al., Cell Adh. Commun. 3, 367 (1995)).

VIII) In vitro Applications

The present invention has a number of in vitro applications. For instance, the present expression system can be used to express a desired protein, for example, any one of the above-listed effector gene expression products, in vitro, and the protein can then be isolated and purified by conventional techniques. Use of the present expression system for the expression of a desired protein in vitro is particularly useful because it reduces the risk that the desired protein is expressed in apoptotic cells. Protein expression in apoptotic cells is undesirable because such cells are known to harbor a large number of proteases which could potentially degrade the expressed protein.

Moreover, it is understood that the present expression system can be used in an number of in vitro assays. For example, the present expression system can be used to compare cellular molecules present in the resting phase GO versus the non-resting phases of the cell cycle. For instance, expression systems can be designed to express a protein that is used to assay the existence or activity of a particular molecule in the GO versus non-GO phases of the cell cycle.

The invention is illustrated in greater details with reference to the examples which follow without being restricted thereto.

### EXAMPLES

### Example 1

### Preparation and Testing of an Expression System Containing a Chimeric Promoter System with a Recombinant Transcription Factor in Endothelial Cells

b) Cloning of the Plasmids Used

The expression system according to the invention is composed of the constructs given hereinbelow: RTA (recombinant transcription activator) construct and reporter construct 1 or 2 with different nucleotide sequences which are sequential downstream.

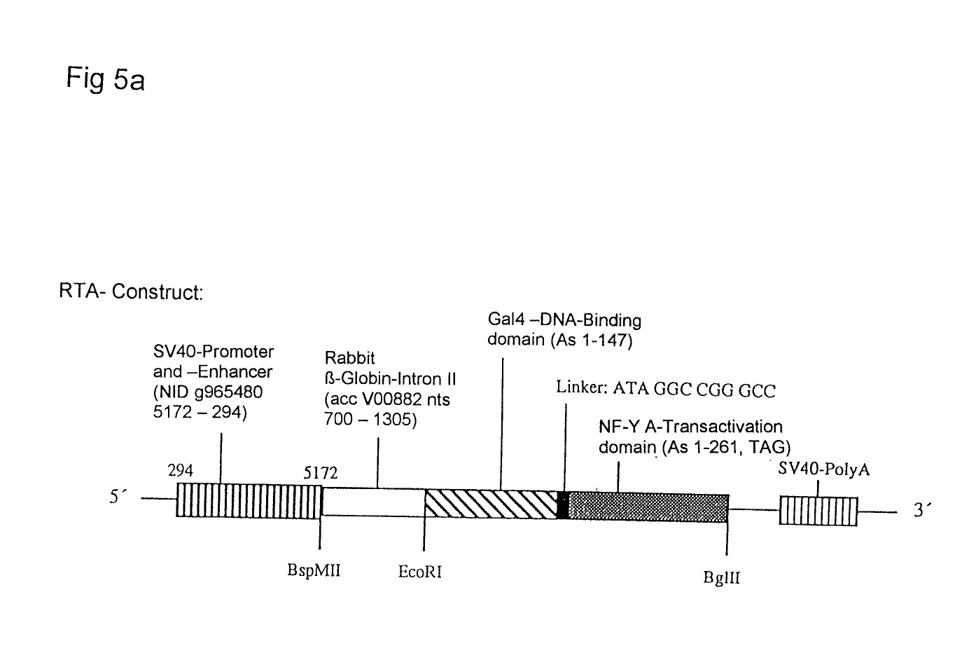

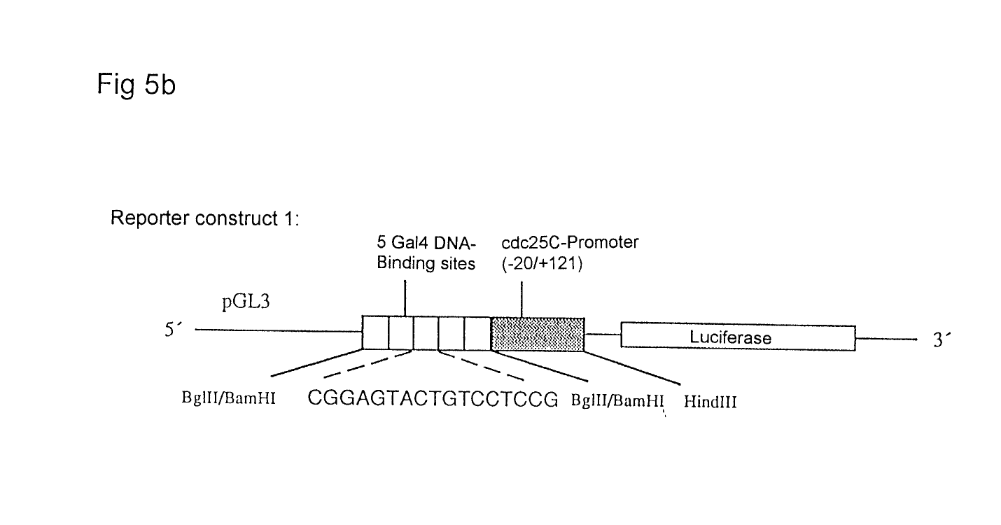

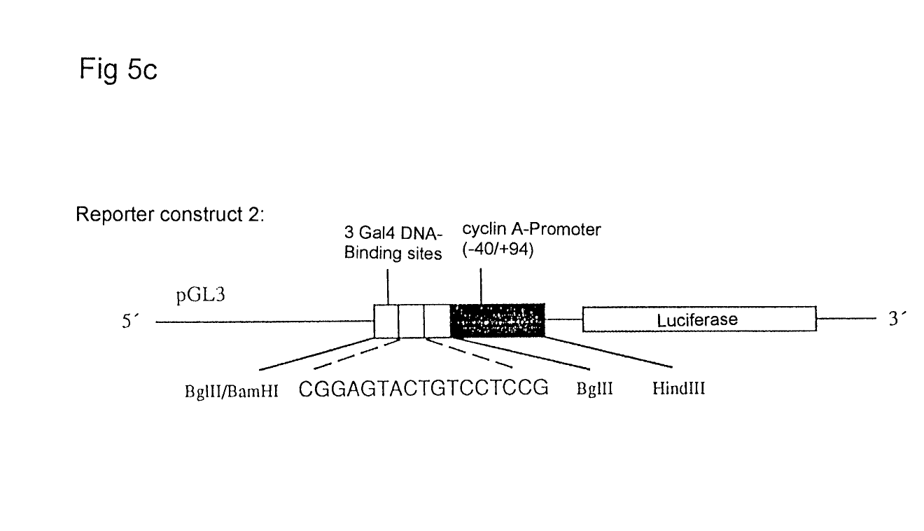

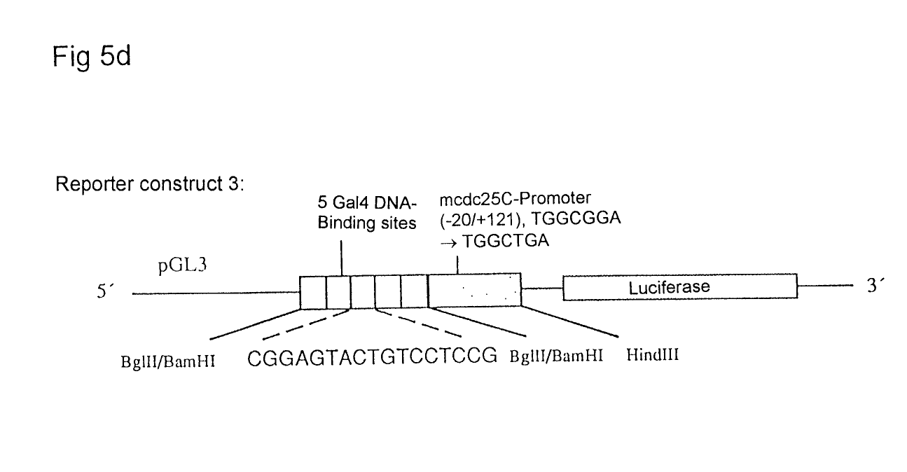

RTA Construct (FIG. 5A)

the SV40 promoter and enhancer (gene bank SV40 circular genome, NID g965480: nucleotides 5172-294)

the rabbit β-globin intron II (gene bank β-globin gene, accession No. V00882: nucleotides 700-1305, van Ooyen et al., Science 206, 337 (1979))

the cDNA for the DNA binding domain of the Gal4 protein [amino acids 1 to 147; Chasman and Kornberg, Mol. Cell Biol. 2916 (1990)]

the linker: ATA GGC CGG GCC (SEQ ID NO: 11)

the cDNA for the transactivation domain of NF-YA [amino acids 1 to 261+stop codon (TAG); Li et al., J. Biol. Chem. 267, 8984 (1992); van Hujisduijnen et al., EMBO J. 9, 3119 (1990); Sinka et al., J. Biol. Chem. 92, 1624 (1995)]

the SV40 poly-A signal (vector pGL3, Promega) (this transcription termination signal is added at the 3′ end of all constructs which are given hereinbelow without being mentioned specifically)

Reporter Construct 1 (FIG. 5B)

5× the binding sequence [nucleotide sequence: 5×5′-CGGAGTACTGTCCTCCG-3′, SEQ ID NO: 6] for the Gal4 protein (Webster et al., Cell 52, 169 (1988))

the basal promoter of cdc25C [nucleotide sequence −20 to +121; Lucibello et al., EMBO J. 14, 132 (1995)]

the cDNA for luciferase; all luciferase constructs are cloned into vector pGL3 (Promega), which contains the SV40 poly-A signal for transcription termination

Reporter Construct 2 (FIG. 5C)

3× the binding sequence [nucleotide sequence: 5×5′-CGGAGTACTGTCCTCCG-3′, SEQ ID NO: 6] for the Gal4 protein (Webster et al., Cell 52, 169 (1988)) the basal promoter of cyclin A (nucleotide sequence −40 to +94; Henglein et al., Proc. Natl Acad. Sci. USA 91, 5490-5494 (1994))

the CDNA for luciferase (pGL3, Promega)

The following constructs were used as controls

Reporter Construct 3 (FIG. 5D)

This corresponds to the reporter construct 1, but the CDE element TGGCGGA in the basal promoter of cdc25C [nucleotide sequence −20 to +121; Lucibello et al., EMBO J. 14, 132 (1995)] was mutated to TGGCtGA.

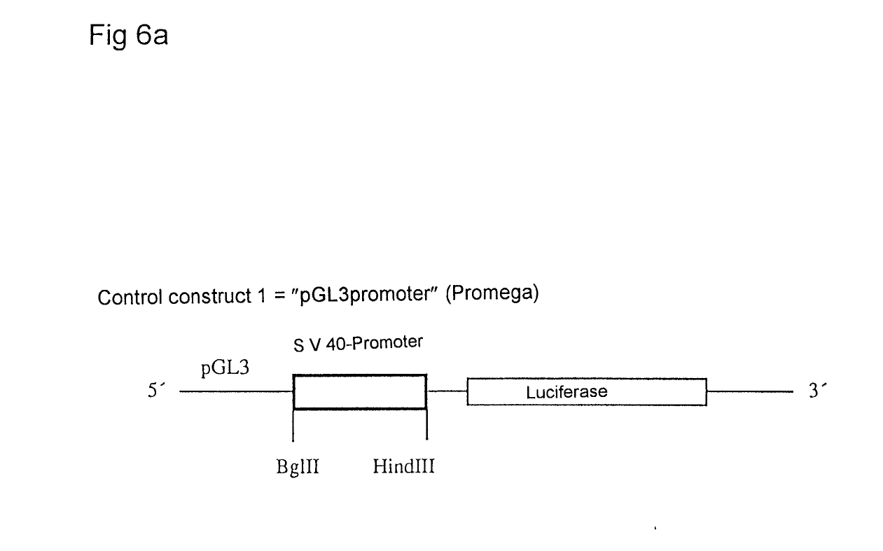

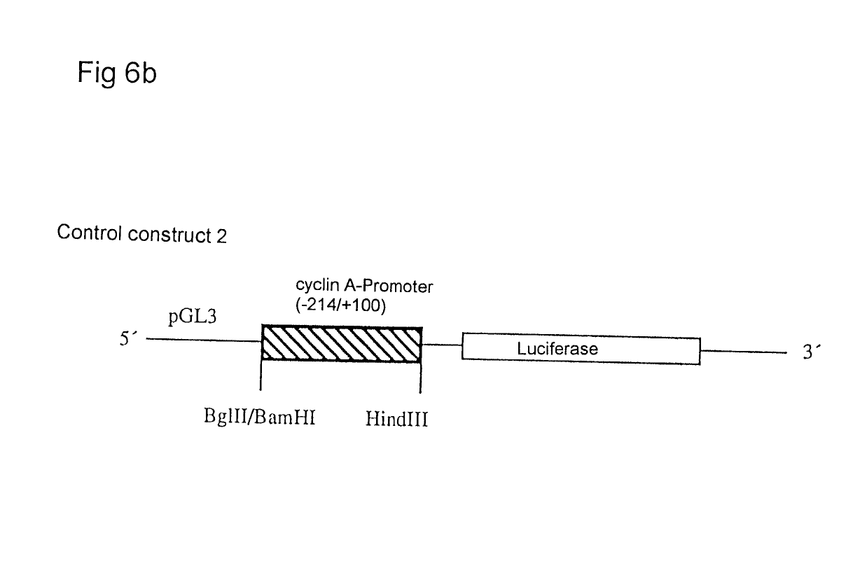

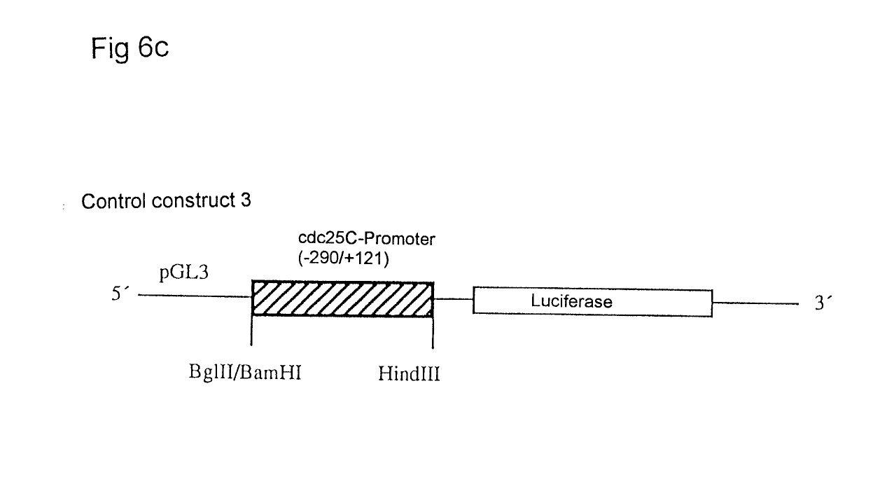

Control Construct 1 (FIG. 6A)

“pGL3promoter” by Promega:

Expression system with the following nucleotide sequences

SV40 promoter

cDNA for luciferase

Control Construct 2 (FIG. 6B)

Expression system with the following nucleotide sequences

cyclin A promoter (-214 to +100, Henglein et al., Proc. Natl Acad. Sci. USA 91, 5490-5494 (1994))

cDNA for luciferase

Control Construct 3 (FIG. 6C)

Expression product with the following nucleotide sequences

cdc25C promoter (-290 to +121, Lucibello et al., EMBO J. 14, 132-142 (1995))

cDNA for luciferase

In order to clone all reporter constructs and control constructs, pGL3 (Promega, luciferase cDNA reporter) was used as vector. The promoter elements cloned into this vector were amplified by means of PCR from human genomic DNA. The oligonucleotides used for this purpose contained in each case an overhang of 4 nucleotides (5-GATC-3), followed by 6 nucleotides with the required restriction cleavage sites (5′ primer: BamHI (GGATCC)/3′ primer: HindIII (AAGCTT) for the control plasmids 1 and 2 and the reporter plasmids 1 and 3; 5′ primer: Bgl II (AGATCT)/3′ primer: HindIII for the reporter plasmid 2) and subsequently 20-25 nucleotides which are complementary to the promoter to be amplified (starting with the position in relation to the transcription start, shown in brackets). The positions shown in brackets refer to the sequence given in the reference cited.

The PCR products were purified using QIAquick™ spin columns (Qiagen) following the manufacturer's instructions, digested with the relevant restriction enzymes (these enzymes are commercially available), separated by agarose gel electrophoresis and then again purified using QIAquick™ spin columns.

Gal4 binding sites were synthesized as oligonucleotides with overhangs required for the relevant restriction cleavage sites (5′: BamHI/3′: Bgl II), purified using SephadexG25 (Pharmacia) and hybridized.

The digested PCR products and the hybridized oligonucleotides were subsequently ligated into the vectors which had been cut in a suitable manner and purified, using T4 DNA ligase (Promega).

All constructs obtained by PCR and by means of using oligonucleotides were sequenced in order to ensure that no mutations were present.

b) Reporter Assays: Transient Transfection, Synchronization and Luciferase Assay

The promoter activity of the constructs described under a) was determined by means of transient transfection, or cotransfection, in endothelial cells followed by measuring luciferase activity. BAECs (bovine aortic endothelial cells) were transfected transiently by the DEAE/dextran method [modified method of Sompayrac et al., PNAS 78, 7575 (1981)]. The luciferase assay was performed as described by Lucibello et al. (EMBO J. 14, 132 (1995)).

8 mg of plasmid were transfected per 3.5 cm dish. In the case of cotransfections, 4+4 μg of plasmid were transfected, and, in the case of the controls, plasmid pUC19 was used for filling up. In order to measure a cell-cycle-dependent promoter activity, proliferating cells (complete medium) were compared with cells which had been arrested in the GI phase of the cell cycle by starving them of methionine for 48 hours.

The control construct 1, which is not cell-cycle-regulated (and which contains the SV40 promoter), was used for standardization (its activity was designated 1).

c) Results

The following results were obtained (measurement values given in brackets represent the relative luciferase activity (standardized with the SV40 promoter=control construct 1) in proliferating cells/relative luciferase activity in G1 cells):

Markedly more luciferase is formed in the endothelial cells transfected with the control constructs 2) and 3) when they proliferate (DNA>2S) than when they are arrested in the G1 phase of the cell cycle (DNA=2S) (control construct 3: >40×; control construct 2: >150×). These constructs acted as controls for the experiments with the expression systems according to the invention.

When the reporter constructs 1) and 2) were cotransfected with the RTA construct, again, higher luciferase activity was demonstrated in proliferating endothelial cells than in G1-arrested endothelial cells (reporter construct 1: 5.5×; reporter construct 2: 8.3×). No difference was observed after cotransfection of the control construct 3) with the RTA construct (1.0×). In each case, the luciferase activity was markedly higher than after transfection of the reporter constructs in question alone.

A pronounced cell cycle regulation of the expression system according to the invention in endothelial cells was thus demonstrated.

### Example 2

### Preparation and Testing of an Expression System Containing a Chimeric Promoter System with a Recombinant Transcription Factor in Melanoma Cells

a) Cloning of the Plasmids Used

The expression system according to the invention consists of the following constructs with different, downstream sequential nucleotide sequences:

RTA Constructs

The RTA (recombinant transcription activator) plasmids encode a fusion protein composed of the Gal4 DNA binding domain and the high-serine, -threonine and -glutamine transactivation domain of the transcription factor NF-YA.

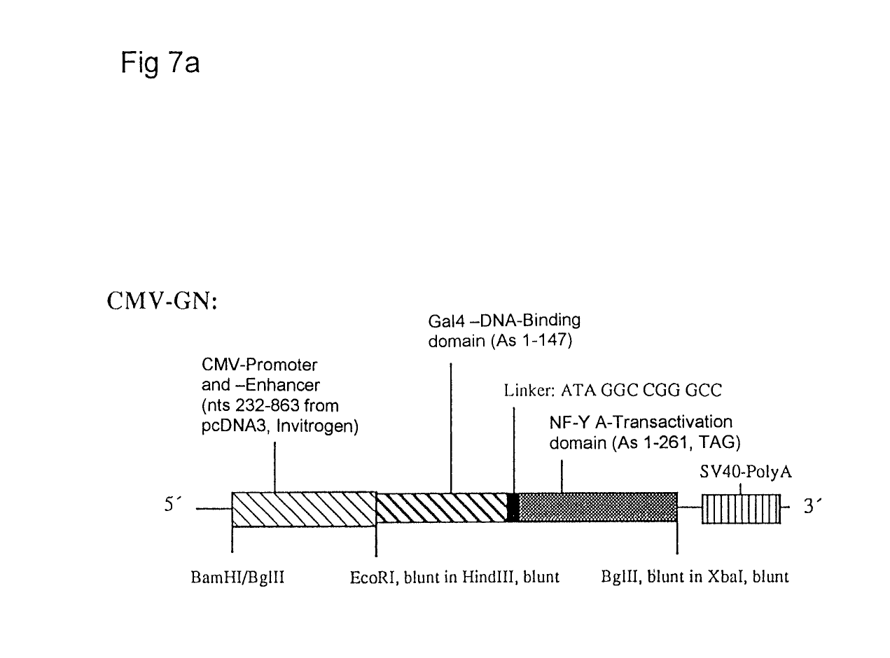

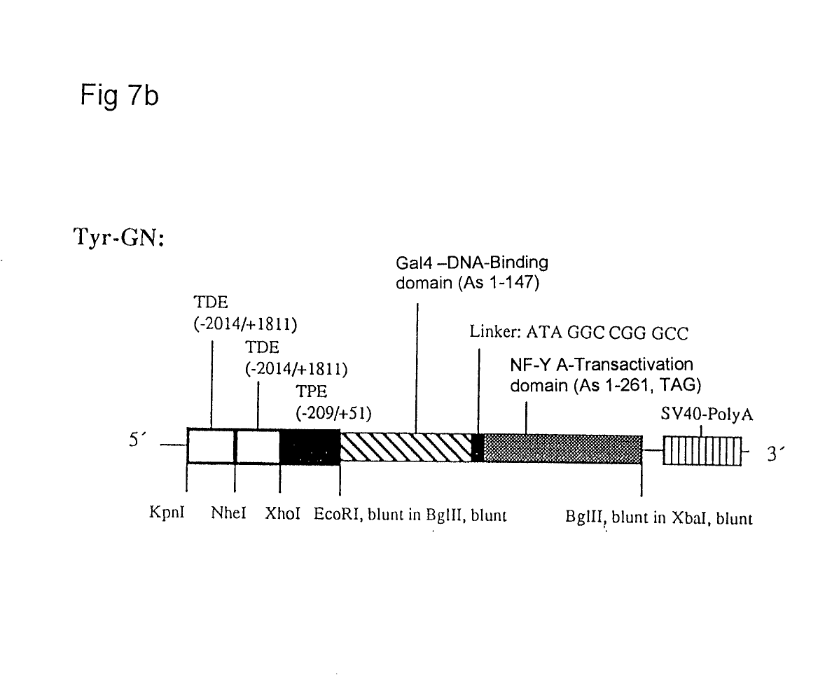

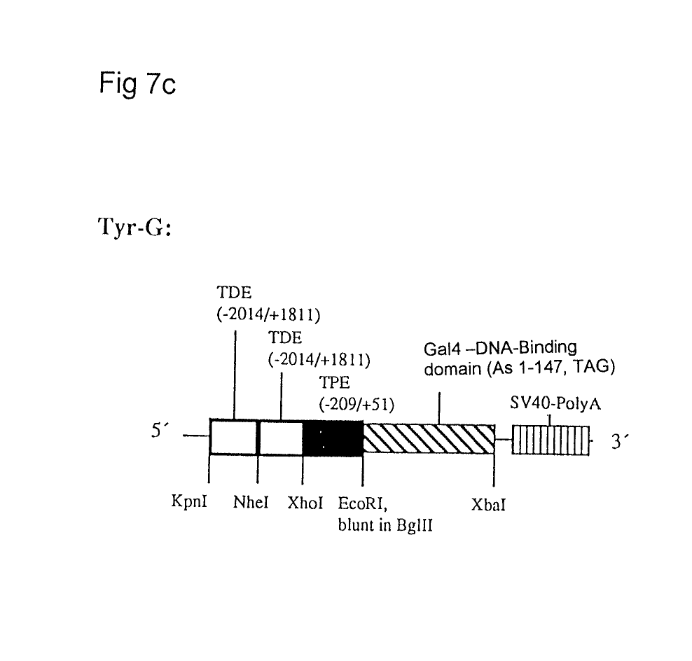

CMV-GN Gal4 (As 1-147), linker: ATA GGC CGG GCC (SEQ ID. NO: 11), mNF-YA (As 1-261+stop codon (TAG)) under the control of the CMV promoter and enhancer (nts 232-863 from pcDNA3, Invitrogen), SV40-PolyA (FIG. 7A) (Li et al., J. Biol. Chem. 267, 8984-8990 (1992))

Tyr-GN Gal4 (As 1-147), linker: ATA GGC CGG GCC (SEQ ID NO: 11), mNF-YA (As 1-261+stop codon (TAG)) under the control of the tyrosinase promoter (see construct Tyr), SV40-PolyA (FIG. 7B) (Li et al., J. Biol. Chem. 267, 8984-8990 (1992))

Tyr-G Gal4-stop (As 1-147+stop codon (TAG)) under the control of the tyrosinase promoter, SV40-PolyA (FIG. 7C)

Reporter Constructs

5G25C identical to reporter construct 1) under I)

5G25CRT7 identical to reporter construct 2) under I)

8GCycA 8×Gal4 DNA binding site +cyclin A promoter (-40/+94; Henglein et al., Proc. Natl Acad. Sci.

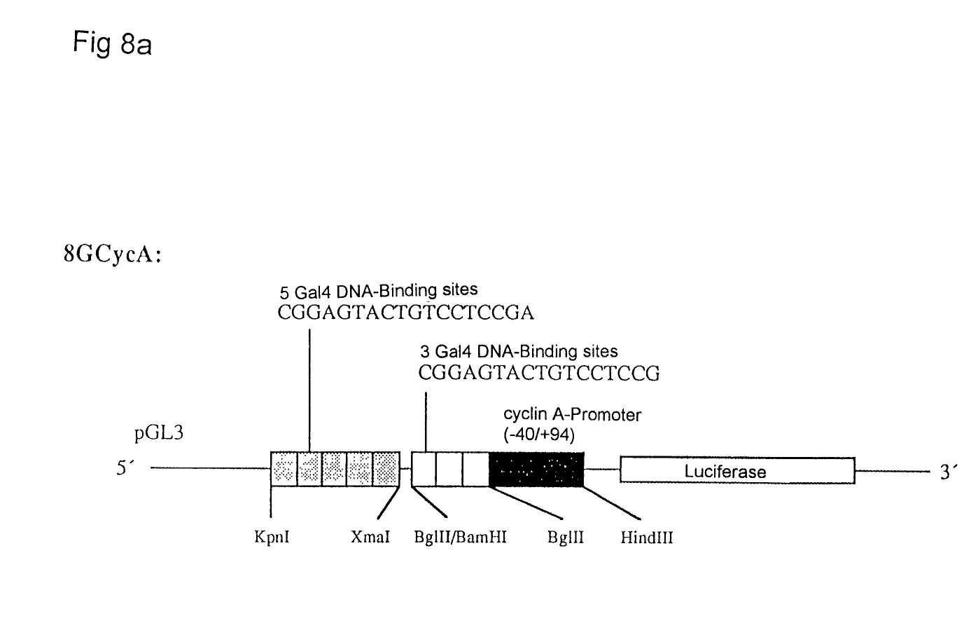

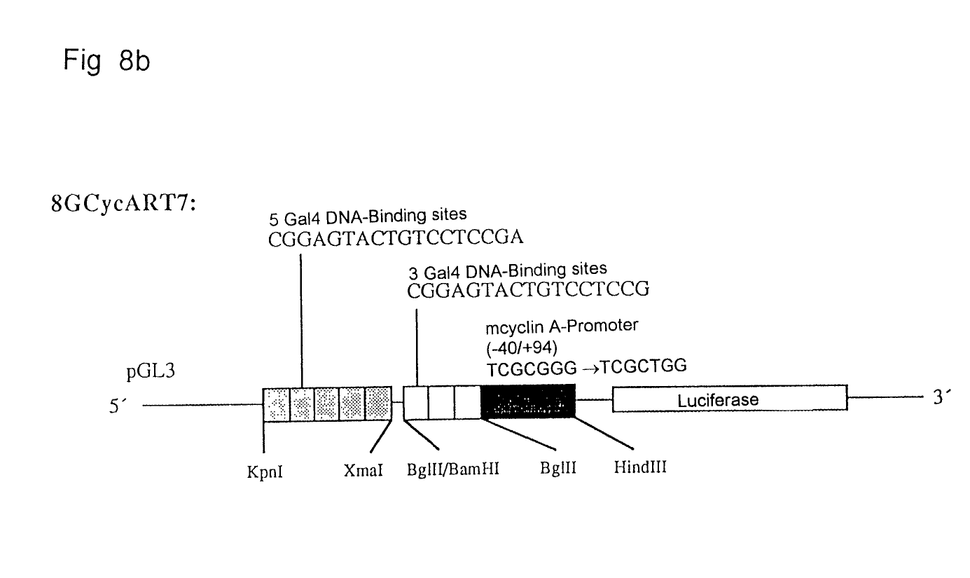

USA 91, 5490-5494 (1994)) (FIG. 8A)

8GCycART71ike 8GCycA, with mutated CDE (TCGCGGG→TCGCtGG, Zwicker et al. EMBO J. 14: 4514, 1995) (FIG. 8B)

Gal4 DNA binding site:

| | |
|---|---|
| 5′-CGGAGTACTGTCCTCCG-3′, | SEQ ID NO: 6 |

Control Constructs

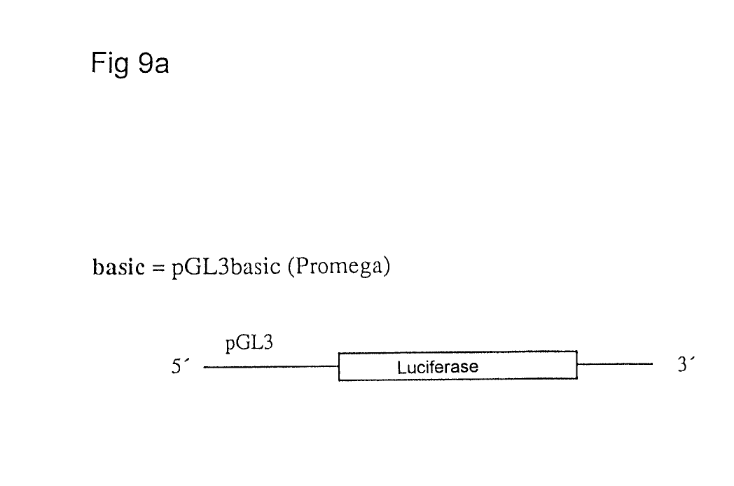

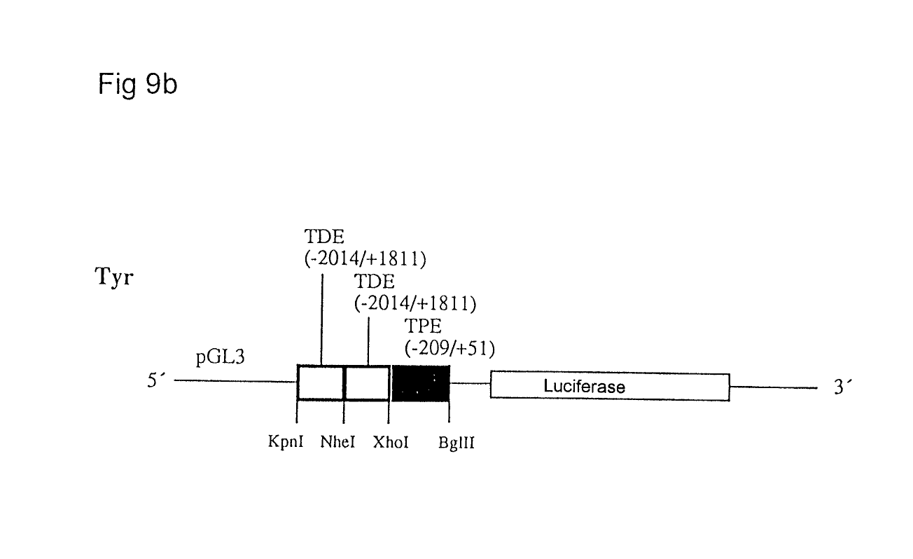

basic =“pGL3basic” (Promega, without promoter or enhancer) (FIG. 9A)

SV40p =“pGL3promoter” (Promega, with the simian virus 40 basal promoter) identical with control construct 1 under I (FIG. 6A)

Tyr tyrosinase promoter: 2× distal element (TDE, −2014/−1811) and 1× proximal element (TPE, −209/+51) (Shibata et al., J. Biol. Chem. 267, 20584 (1992)), cDNA for luciferase (pGL3, Promega) (FIG. 9B)

cdc25C cdc25C promoter (−290/+121) (Lucibello et al., EMBO J. 14, 132-142 (1995)), cDNA for luciferase (pGL3, Promega); identical with control construct 3 under I (FIG. 6C)

cycA cyclin A promoter (−214/+100) (Henglein et al., Proc. Natl Acad. Sci. USA 91, 5490-5494 (1994)), cDNA for luciferase (pGL3, Promega); identical with control construct 2 under I (FIG. 6B)

pGL3 (Promega, luciferase cDNA reporter) was used as the vector in all reporter constructs and control constructs. The promoter elements cloned into this vector were amplified by means of PCR from human genomic DNA. The oligonucleotides used for this purpose contained in each case an overhang of 4 nucleotides (GATC), followed by 6 nucleotides with the required restriction cleavage sites (BamHI (GGATCC)/HindIII (AAGCTT) for control plasmids cdc25C and cycA and the reporter plasmids 5G25C and 5G25CRT7; Kpnl (GGTACC)/Nhel (GCTAGC) and NheI/XhoI (CTCGAG) for TDE and XhoI/BglII (AGATCT) for TPE, BglII/HindIII for the reporter plasmids 8GCycA and 8GCycART7 and subsequently 20-25 nucleotides which are complementary to the promoter to be amplified (starting with the position relative to the transcription start which is shown in brackets). The positions given in brackets refer to the sequence mentioned in the cited reference.

The PCR products were purified using QIAquick™ spin columns (Qiagen) following the manufacturer's instructions, digested with the relevant restriction enzymes (these enzymes are commercially available), separated by agarose gel electrophoresis and then again purified using QIAquick™ spin columns.

Gal4 binding sites were synthesized as oligonucleotides with overhangs required for the relevant restriction cleavage sites (KpnI (top: 5′; bottom: 3′)/XhoI (top:5′; bottom: 3′) or BamHII (top: 5′; bottom: 3′)/BglII (top: 5′; bottom: 3′)), purified using SephadexG25 (Pharmacia) and hybridized.

The digested PCR products and the hybridized oligonucleotides were subsequently ligated into the vectors which had been cut in a suitable manner and purified, using T4 DNA ligase (Promega).

All constructs obtained by PCR and by using oligonucleotides were sequenced in order to ensure that no mutations were present.

b) Reporter Assays: Transient Transfection, Synchronization and Luciferase Assay

The promoter activity of the constructs described under a) was determined by means of transient cotransfection in melanocytes (MeWo, human), fibroblasts (3T3, murine) and prostate carcinoma cells (PC-3, human) and subsequent measurement of the luciferase activity. The cells were transfected transiently with DOTAP (Boehringer, Mannheim) following the manufacturer's instructions.

The luciferase assay was carried out as described by Lucibello et al. (EMBO J., 14, 132 (1995)). Per 3.5 cm dish, 1 mg of reporter+2 mg of RTA plasmid were transfected with 6 ml of DOTAP, pUC19 plasmid being employed in place of the RTA plasmid in the case of the controls.

In order to measure a cell-cycle-dependent promoter activity, proliferating cells (complete medium) were compared with the cells which had been arrested in the G1 phase of the cell cycle. The construct SV40p, which is not cell-cycle-regulated, was used for standardization (its activity was designated 1). The cells were synchronized in the G1 phase after transfection by starving them of methionine for 60 hours.

In order to measure the cell-type-specific promoter activity, the luciferase activities of the various cell types were standardized and then compared with the values for the ubiquitous SV40 promoter (where SV40p=1).

c) Results

Table 1 shows a pronounced cell-type-specificity of the system: (1) the 8GCycA construct shows only little activity which is within the range of the activity of the basic vector “basic”, (2) cotransfection of the CMV-GN construst results in pronounced activation in all 3 cell lines, (3) cotransfection of the Tyr-GN construct leads to specific activation only in the target cells, i.e. in the melanoma cells, by selective expression of Gal-NF-Y fusion protein, and (4) cotransfection of the Tyr-G construct only leads to very weak activation, i.e. the activation in (3) can be attributed to the NF-YA transactivation domain.

The specifity of the system 8GCycA+Tyr-GN is 58 (comparison MeWo : PC-3) or 73 (comparison MeWo : 3T3), with very weak activity in non-target cells.

The values in Table 2 confirm that the activity of the system is cell-cycle-regulated:

the cyclin A promoter demonstrates a cell cycle regulation which is increased by a factor of 26 (activity of proliferating MeWos : MeWos activity in G1, positive control)

the system 8GCycA +Tyr-GN demonstrates a cell cycle regulation which is increased by a factor of 22.5 (and is thus almost equally well regulated as the cyclin A wild-type promoter, the activity in proliferating cells being almost identical) and

a mutation of the CDE element (RT7) results in a drastically increased activity in G1 cells and thus to a cell cycle regulation which is decreased by a factor of 5. The cell cycle regulation can therefore be attributed mainly to the CDE/CHR-mediated repression in G1 (caused by the CDE/CHR-binding repressor, which represses, in G1 cells, the transactivation caused by NF-YA). The remaining cell cycle regulation of the RT7 mutant can be attributed to the cell cycle regulation of the tyrosinase promoter itself, which is also low (factor 4.2).

Tables 3 and 4 demonstrate that, when using the cdc25C-CDE/CHR element, the system not only exhibits pronounced cell type specificity (factor 3.9), but also cell cycle regulation (factor 8.4).

It was thus possible to demonstrate by way of example the tissue-specific expression of a Gal-NF-Y fusion protein which controls the expression of a gene both tissue-specifically and in a proliferation-dependent manner via Gal4 DNA binding sites upstream of a CDE/CHR element. When use was made of the melanocyte-specific tyrosinase promoter and the cyclin A-CDE/CHR element, cell cycle regulation was increased by a factor of >20, while cell type specificity was increased by a factor of >50. The activity of the system in proliferating target cells is similar to the activity of the wild-type cyclin A promoter, and in non-proliferating target cells and in non-target cells scarcely not higher than that of the basic vector pGL3basic.

### Example 3

### In vivo Experiments

In order to find out whether the transcription level which is achieved by the promoter system according to the invention is sufficient for achieving a biological effect, a TNF-α cytolysis assay was carried out in vitro. This assay, which measures cytotoxic effects on the TNF-α-sensitive cell line L 929, was performed using, as the medium, MeWo cells which had been cotransfected with activator (Tyr-GN) and effector (Gal Cyc ATNF) constructs. In order to construct Gal Cyc ATNF, the luciferase cDNA of Gal Cyc A was replaced by the TNF-α cDNA from plasmid pAS3 (obtained from M. Clauss, Max Planck Institut, Bad Nauheim, Germany). pAS3 contains the murine TNF-α cDNA cloned into the PstI/EcoRI restriction site of the vector pBluescript II SK.

To obtain the highest possible transfection rate in the TNFα bioassay, MeWo cells were used together with lipofectin (Life Technologies) in accordance with the manufacturer's instructions. One microgram Gal Cyc ATNF and 1 μg pUC19 or Tyr-GN were mixed with 10 μl of lipofectin in OptiMEM and the cells incubated herewith for 6 hours.

The MeWo cells were cotransfected with Gal Cyc ATNF/pUC19 or Gal Cyc ATNF/Tyr-GN in three parallel batches. 24 hours after transfection, the medium was replaced and, after a further 24 hours, collected. The culture supernatants were tested for TNFα bioactivity by determining their cytotoxicity on the transformed mouse fibroblast cell line L 929. Such L 929 cells were seeded in microtiter plates at a density of 4×10⁴ cells per well. After 16 hours, serial dilutions of mouse TNFA in conditioned medium of untransfected MeWo cells and the supernatants of the transfected cells and in each case actinomycin D were added to an end concentration of 1 μg/ml. After 24 hours, the remaining L 929 cells were fixed, stained with crystal violet, and the adhering dye was quantified using an ELISA reader at a wavelength 8=450 nm.

48 hours after transfection, cytolysis was around 60% (corresponding to ^{˜}1.0 ng/ml TNFα in the supernatant). In contrast, supernatants of cells which had been transfected only with the Gal Cyc A effector construct showed only a negligibly small proportion of dead cells (<2%). These results demonstrated that the transcription rate achieved by means of the promoter system according to the invention is suitable for achieving a pronounced biological effect.

The contents of Federal Republic of Germany priority application 19831420.5, filed Jul. 14, 1998, are hereby incorporated by reference in their entirety. Furthermore, all documents referred to herein are hereby incorporated by reference win their entirety.

TABLE 1

| | Luciferase activity | | |
|---|---|---|---|
| | (RLUs, SV40p = 1) | | |
| Constructs | MeWo | 3T3 | PC-3 |
| Basic | 0.01 | 0.01 | 0.07 |
| SV40p | 1.00 | 1.00 | 1.00 |
| Tyr | 57.6 | 0.03 | 0.05 |
| 8GcycA | 0.04 | 0.02 | 0.06 |
| 8GcycA + CMV-GN | 3.39 | 0.42 | 1.35 |
| 8GcycA + Tyr-GN | 2.92 | 0.04 | 0.05 |
| 8GcycA + Tyr-G | 0.18 | 0.02 | 0.02 |

TABLE 2

Luciferase activity
(RLUs, SV40p = 1)

| | MeWo | MeWo |
|---|---|---|
| Constructs | proliferating | G1 |
| Basic | 0.01 | 0.05 |
| SV40p | 1.00 | 1.00 |
| Tyr | 57.60 | 13.60 |
| CycA | 3.38 | 0.13 |
| 8GcycA | 0.01 | 0.07 |
| 8GcycA + Tyr-GN | 2.70 | 0.12 |
| 8GcycART7 | 0.04 | 0.05 |
| 8GcycART7 + Tyr-GN | 7.25 | 1.44 |

TABLE 3

Luciferase activity
(RLUs, SV40p = 1)

| Constructs | MeWo | 3T3 |
|---|---|---|
| SV40p | 1.00 | 1.00 |
| 5G25C | 0.05 | 0.14 |
| 5G25C + CMV-GN | 6.50 | 7.13 |
| 5G25C + Tyr-GN | 5.46 | 1.39 |

TABLE 4

Luciferase activity
(RLUs, SV40p = 1)

| | MeWo | MeWo |
|---|---|---|
| Constructs | proliferating | G1 |
| SV40p | 1.00 | 1.00 |
| Cdc25C | 1.08 | 0.11 |
| 5G25C | 0.05 | 0.22 |
| 5G25C + Tyr-GN | 5.46 | 0.65 |
| 5G25CRT7 | 0.00 | 0.17 |
| 5G25CRT7 + Tyr-GN | 3.98 | 2.58 |

1. A nucleic acid construct which comprises the following components:
Component a): at least one promoter;
Component b): a nucleic acid sequence encoding at least one recombinant transactivator whose expression is activated by component a) and wherein said transactivator comprises:
component b1): a nucleic acid sequence encoding a DNA-binding domain; and
component b2): a nucleic acid sequence encoding a transactivation domain comprising glutamine, serine and threonine;
Component c): at least one nucleic acid sequence which binds to the expression product of component b);
Component d): at least one promoter which comprises the CDE-CHR element or the E2FBS-CHR element and whose 5′ end is linked to the 3′ end of component c); and
Component e): at least one effector gene whose transcription is activated by the expression product of component b) binding to component c).

2. The nucleic acid construct of claim 1, wherein at least one component c) is linked to the 5′ end of component a).

3. The nucleic acid construct of claim 1, further comprising component b′), wherein component b′) comprises:
component b1) linked to a component b3) wherein component b3) comprises a nucleic acid sequence that encodes a protein A which binds to a coupling substance f); and
component b2) linked to a component b4) wherein component b4) comprises a nucleic acid sequence that encodes a protein B which binds to said coupling substance f); and wherein
said coupling substance f) links the expression products of components b1), b3), b4) and b2) thereby forming an operative recombinant transactivator.

4. The nucleic acid construct of claim 1, further comprising component b″), wherein component b″) encodes a transactivator and comprises:
components b1) and b2) linked to a component b5) wherein component b5) comprises a nucleic said sequence that encodes at least one binding protein for a cellular regulatory protein, wherein the binding of said cellular regulatory protein to said binding protein inhibits said transactivator expressed by component b″).

5. The nucleic acid construct of claim 1, wherein two or more are effector genes are linked to each other by an internal ribosome entry site (IRES) sequence or by said components c) and d).

6. The nucleic acid construct of claim 1, wherein a nuclear localization signal is linked to component b).

7. The nucleic acid construct of claim 1, wherein said promoter of component a) comprised:
a promoter selected from the group consisting of an RNA polymerase III or RNA polymerase II promoter, CMV promoter and enhancer, and SV40 promoter; or
a viral promoter and activator sequence selected from the group consisting of an HBV, HCV, HSV, HPV, EBV, HTLV, and HIV promoter and activator sequences;
a cell-cycle-specifically activatable promoter selected from the group consisting of a cdc25C, cyclin A, cdc2 (cdk-1), Bmyb, DHFR, and E2F-1 gene promoters and binding sequences for transcription factors which occur or are activated in a cell-proliferation-dependent manner; or
a cell-specifically-activatable promoter selected from the group consisting of promoters which are cell-specifically activatable in endothelial cells, connective tissue cells, muscle cells, glia cells, hematopoietic cells, lymphocytes, macrophages, synovial cells, leukemia cells, tumor cells, cells of the gastrointestinal mucosa, of the kidneys, of the respiratory organs, of the sexual organs and of the lower urinary tract; or
a metabolically activatable promoter.

8. The nucleic acid construct of claim 1, wherein component b1) is selected from the group consisting of the DNA-binding domains of Gal4 protein, LexA protein, lac repressor protein, tetracycline repressor protein and ZFHD1 protein.

9. The nucleic acid construct of claim 1, wherein component b2) comprises at least 20× glutamine, 10× serine and 10× threonine.

10. The nucleic acid construct of claim 9, wherein component b2) comprises a transactivation domain selected from the group consisting of the transactivation domains of Oct-2, SP1 and NFY-1.

11. The nucleic acid construct of claim 3, wherein at least one of said proteins A and B is an antibody or an antibody fragment.

12. The nucleic acid construct of claim 11, wherein at least one of said proteins A and B is a single-chain Fv fragment comprising a variable chain and a light chain which are linked covalently by a peptide sequence.

13. The nucleic acid construct of claim 3, wherein at least one of said proteins A and B comprise a binding domain of a binding protein wherein said binding domain binds to said coupling substance f).

14. The nucleic acid construct of claim 3, wherein said coupl ing substance f) is a drug.

15. The nucleic acid construct of claim 3, wherein said coupling substance f) is capable of penetrating a cell membrane.

16. The nucleic acid construct of claim 14, wherein said coupling substance f) is selected from the group consisting of rapamycin, FK506, cyclosporin A, methotrexate, folic acid, retinoic acid, penicillin, 4-hydroxy-tamoxifen, tamoxifen, tetracycline and a tetracycline/isopropyl-β-D-thiogalactoside conjugate.

17. The nucleic acid construct of claim 4, wherein said component b5) encodes a cellular binding protein or a fragment thereof.

18. The nucleic acid construct of claim 17, wherein said cellular binding protein or fragment thereof binds to a cellular regulatory protein which is selected from the group consisting of p53, pRb, pl30, Max, MAD, VHL, cdk-4, MTS-1 (pl6), WT-1, SMAD-2, and DPC-4.

19. The nucleic acid construct of claim 17, wherein said component b5) encodes a cellular binding protein selected from the group consisting of E2F 1, E2F 2, E2F 3, E2F 4, E2F 5, cyclin D1, cyclin D2, cyclin D3, cyclin C, cyclin A, cyclin E, Myc, transcription factor PU.1 or Elf-1, elongin B, elongin C, p14, p15, p16, p18, p21, p27, p53, Myc, cdk-4, DPC-4 and SMAD-2.

20. The nucleic acid construct of claim 17, wherein said component b5) encodes a viral binding protein or a fragment thereof.

21. The nucleic acid construct of claim 20, wherein said viral binding protein or fragment thereof binds to a cellular regulatory protein selected from the group consisting of p53, pRb (p110), NFKB, p130, CBF-1, lyn tyrosine kinase, bak and bax.

22. The nucleic acid construct of claim 20, wherein said component b5) encodes a viral binding protein selected from the group consisting of IE 84 of CMV, E1B (55 kD) of AV, EBNA-5 of EBV, BHFR of EBV, E6 of HPV 16 or HPV 18, x protein of HBV, T antigen of SV40, E1A of AV, EBNA-2 of EBV, EBNA-1 of EBV, E7 of HPV, Tax of HIV, LMP-1 of EBV, LMP-2A or LMP-2B of EBV, E1B (16 kD) of AV, and E1B (10 kD) of AV.

23. The nucleic acid construct of claim 4, wherein component b5) encodes an antibody or a fragment thereof.

24. The nucleic acid construct of claim 1, wherein said component c) comprises a nucleic acid sequence selected from the group consisting of a binding sequence for the Gal4 protein, a binding sequence for the LexA protein, a binding sequence for the Lac I repressor protein, a binding sequence for the tetracycline operator and a binding sequence for the ZFHD-1 protein.

25. The nucleic acid construct of claim 1, wherein said component d) comprises a CDE-CHR element selected from the group consisting of a cdc25C gene, a cdc2 (cdk-1) gene and a cyclin A gene CDE-CHR element.

26. The nucleic acid construct of claim 1, wherein said component d) comprises a Bmyb gene E2FBS-CHR element.

27. The nucleic acid construct of claim 1, wherein said component e) encodes an active substance selected from the group consisting of cytokines, chemokines or growth factors, proteins with an antiproliferative or cytostatic or apoptotic action, inflammatory or immunosuppressive proteins, antibodies, antibody fragments, angiogenesis inhibitors, peptide hormones, coagulation factors, coagulation inhibitors, fibrinolytic proteins, peptides or proteins which are effective on the blood circulation, blood plasma proteins and antigens of pathogens or of cells or of tumors.

28. The nucleic acid construct of claim 1, wherein said component e) encodes an enzyme which cleaves a prodrug into a drug.

29. The nucleic acid construct of claim 1, wherein said component e) encodes a ligand/active substance fusion protein or a ligand/enzyme fusion protein, the ligand being selected from the group consisting of cytokines, growth factors, antibodies, antibody fragments, peptide hormones, mediators and cell adhesion molecules.

30. The nucleic acid construct of claim 1, wherein said nucleic acid sequences are DNA sequences.

31. The nucleic acid construct of claim 1, wherein the nucleic acid construct is inserted into a vector.

32. The nucleic acid construct of claim 31, wherein said vector is a plasmid vector.

33. The nucleic acid construct of claim 31, wherein said vector is a viral vector.

34. A method of treating a disease comprising administering externally, perorally, intravesicularly, nasally, intrabronchially or into the gastrointestinal tract, or injecting into an organ, into a body cavity, into the muscle system, subcutaneously or into the blood circulation said nucleic acid construct of claim 1.

35. An isolated cell, which comprises the nucleic acid construct of claim 1.

36. The isolated cell of claim 35, wherein said cell is selected from the group consisting of endothelial cells, lymphocytes, macrophages, hematopoietic cells, fibroblasts, muscle cells, liver cells, kidney cells, epithelial cells of the gastrointestinal tract, of the respiratory system, of the lower urinary tract, of the sexual organs, of the skin, glia cells, cells of the nervous system, tumor cells and leukemia cells.

37. A process for the preparation of the nucleic acid construct of claim 1 comprising ligating together components a)-e).

38. A method of treating a disease comprising administering externally, intravesicularly, nasally, intrabronchially, orally or into the gastrointestinal tract, or injecting into an organ, into a body cavity, into the muscle system, subcutaneously or into the blood circulation at least one of said cells of claim 35.

39. The nucleic acid construct of claim 7, wherein said metabolically activatable promoter is a hypoxia-inducible enhancer.

40. The nucleic acid construct of claim 7, wherein said binding sequences are monomers or multimers of the Myc E box.
